(19) United States
(12) Patent Application Publication
(10) Pub. No.: US 2006/0205819 A1
(43) Pub. Date: Sep. 14, 2006

(54) Methods of treatment inflammatory bowel with lxr agonists

(76) Inventors: Yukio Goto, Tokyo (JP); Hideo Kikkawa, Ibaraki (JP); Mine Kinoshita, Ibaraki (JP)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/566,637

(22) PCT Filed: Jul. 27, 2004
(86) PCT No.: PCT/EP04/08426

§ 371 Date: Jan. 27, 2006

Related U.S. Application Data
(60) Provisional application No. 60/490,614, filed on Jul. 28, 2003.

(51) Int. Cl. A61K 31/195; A61K 31/18

(52) U.S. Cl. 514/567; 514/602

(57) ABSTRACT

The present invention relates generally to the use of LXR agonists in the prevention and/or treatment of inflammatory bowel diseases.

### FIELD OF THE INVENTION

The present invention relates generally to the use of LXR agonists in the prevention and/or treatment of inflammatory bowel diseases.

### BACKGROUND OF THE INVENTION

LXRα and LXRβ (collectively LXR) are nuclear hormone receptors that regulate the metabolism of several important lipids, including cholesterol (1). The nucleotide and amino acid sequences of LXRα are shown in FIGS. 1 and 2 (SEQ ID NOs: 1 and 2), respectively. The nucleotide and amino acid sequences of LXRβ are shown in FIGS. 3 and 4 (SEQ ID NOs:3 and 4), respectively. The LXRs regulate the expression of target genes by binding to short stretches of DNA, termed LXR response elements (LXREs), as heterodimers with the retinoid X receptors (RXR)(2-5). LXREs have been identified in the regulatory regions of a number of genes involved in cholesterol homeostasis including CYP7A1 (6), which catalyses the first and rate-limiting step in bile acid biosynthesis, the cholesterol ester transport protein (7), the transcription factor SREBP-1C (8,9), and apolipoprotein E (apoE)(10). LXREs have also been identified in the genes encoding the ATP binding cassette transporters (ABC) A1 and G1(11-15), which mediate the efflux of phospholipids and cholesterol from macrophages, intestinal enterocytes and other cell types.

Currently, patients with elevated levels of cholesterol are treated using the compounds that inhibit the body's endogenous cholesterol synthesis. As important components of the complex system that regulates cholesterol levels in the body, the LXRs have also been proposed as targets for the prophylaxis and treatment of hypercholesteraemia (raised levels of plasma cholesterol) and its associated atherosclerotic diseases.

Inflammatory bowel disease (IBD) is a group of chronic disorders that cause inflammation in the small and large intestine. IBD includes Crohn's disease and ulcerative colitis. Further, IBD can also include inflammatory colitis caused by bacteria, ischemia, radiation, drugs or chemical substances. The use of agonists of LXR and their pharmaceutical formulations to reverse cholesterol transport and treat atherosclerotic cardiovascular diseases have been reported. However, until Applicants' present discovery, the use of LXR agonists for treating or preventing IBD has not been reported.

### SUMMARY OF TH INVENTION

In one aspect, the present invention provides a method of treating or preventing IBD in a mammal, including, but not limited to Crohn's disease, ulcerative colitis, and inflammatory colitis caused by bacteria, ischemia, radiation, drugs or chemical substances; comprising, administering a therapeutically effective amount of LXR agonistl, or a pharmaceutically acceptable salt, solvate, or physiologically functional derivative thereof.

In further aspect, the invention also relates to a pharmaceutical composition comprising a therapeutically effective amount of LXR agonist, or a pharmaceutically acceptable salt, solvate, or physiologically functional derivative thereof, and a pharmaceutically acceptable carrier for the treatment or prevention of IBD in a mammal, including, but not limited to Crohn's disease, ulcerative colitis, and inflammatory colitis caused by bacteria, ischemia, radiation, drugs or chemical substances.

Yet in a further aspect, the present invention relates to the use of a LXR agonist in the preparation of a medicament for the treatment or prevention of IBD in a mammal, including, but not limited to Crohn's disease, ulcerative colitis, and inflammatory colitis caused by bacteria, ischemia, radiation, drugs or chemical substances.

### BRIEF DESCRIPTION OF THE DRAWINGS

FIG. 1 shows the nucleotide sequence of human LXRα (SEQ ID NO:1) from Genebank, accession NM_{—}005693.

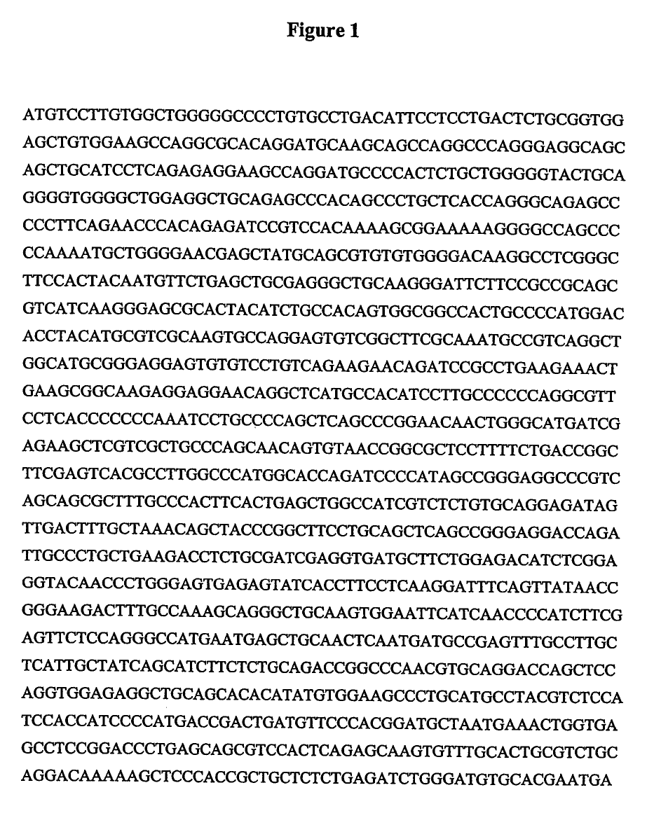

FIG. 2 shows the deduced amino acid sequence of human LXRα (SEQ ID NO:2) from Genebank accession NP_{—}005684.

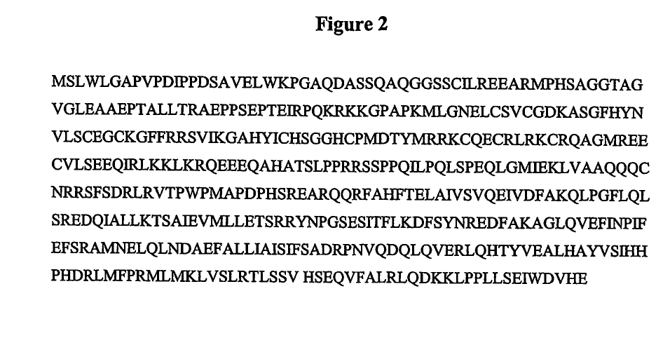

FIG. 3 shows the nucleotide sequence of human LXRβ from Genebank accession (SEQ ID NO:3) from Genbank accession XM_{—}046419.

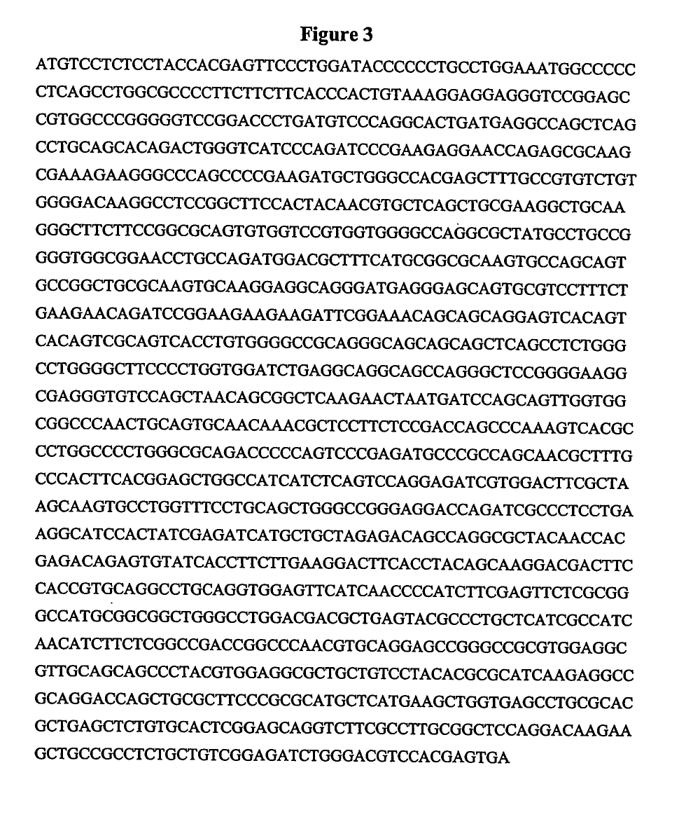

FIG. 4 shows the deduced amino acid sequence of human LXRβ (SEQ ID NO:4) from Genebank accession XP_{—}046419.

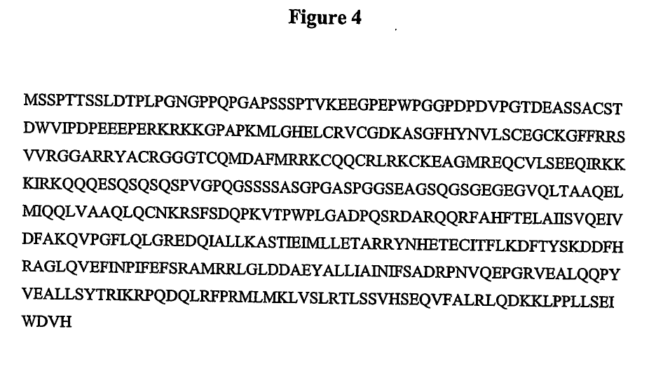

### DETAILED DESCRIPTION OF THE INVENTION

The term “LXR agonist” means any compound that enhances the biological activities of LXRα and/or LXRβ. LXR agonists are well known. Preferred LXR agonists of the present invention are selected from compounds of formulas (I), (II), (E), (I), and (V). The compounds of formulas (I), (II), (III), (IV), and (V) are described in more detail below. Other examples of LXR agonists which form part of instant invention are described in:

WO2002090375 published Nov. 14, 2002;

WO2002058532 published Aug. 1, 2002;

WO200211708 published Feb. 14, 2002;

WO200160818 published Aug. 23, 2001;

WO200115676 published Mar. 8, 2001;

WO200103705 published Jan. 18, 2001; and

WO200066611 published Nov. 9, 2000.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “physiologically functional derivative” refers to any pharmaceutically acceptable derivative of a compound of the present invention, for example, an ester or an amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry And Drug Discovery, 5th Edition, Vol 1: Principles and Practice, which is incorporated herein by reference to the extent that it teaches physiologically functional derivatives.

As used herein, the term “solvate” refers to a complex of variable stoichiometry formed by a solute and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

International Patent Application WO 00/54759 (Tularik Inc. US) discloses compounds of formula (I): 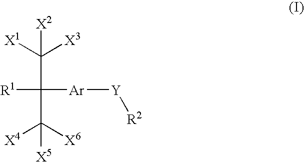
wherein:
Ar represents an aryl group; R¹ is —OH, —O—(C₁-C₇)alkyl, —OC(O)—(C₁-C₇)alkyl, —O—(C₁-C₇)heteroalkyl, —OC(O)— (C₁-C₇)heteroalkyl, —CO₂H, —NH₂, —NH(C₁-C₇)alkyl, —N((C₁-C₇)alkyl)₂ or —NH—S(O)₂—(C₁-C₅)alkyl;
R² is (C₁-C₇)alkyl, (C₁-C₇)heteroalkyl, aryl and aryl(C₁-C₇)alkyl;
X¹, X², X³, X⁴, X⁵ and X⁶ are each independently H, (C₁-C₅)alkyl, (C₁-C₅)hetroalkyl, F or Cl, with the proviso that no more than three of X¹ through X⁶ are H, (C₁-C₅)alkyl or (C₁-C₅)heteroalkyl; and
Y is —N(R¹²)S(O)ₘ—, —N(R¹²)S(O)ₘN(R¹³)—, —N(R¹²)C(O)—, —N(R¹²)C(O)N(R¹³)—, —N(R¹²)C(S)— or —N(R¹²)C(O)O—, wherein R12 and R13 are each independently hydrogen, (C₁-C₇)aryl, (C₁-C₇)heteroalkyl, aryl and aryl(C₁-C₇)alkyl, and optionally when Y is —N(R¹²)S(O)ₘ— or —N(R¹²)S(O)ₘN(R¹³)—, R¹² forms a five, six or seven-membered ring fused to Ar or to R² through covalent attachment to Ar or R², respectively. In the above Y groups, the subscript m is an integer of from 1 to 2, as being useful as agonists of LXR and their use in pharmaceutical formulations to reverse cholesterol transport and treat atherosclerotic cardiovascular diseases and related diseases.

With respect to the compounds of formula (I) the term “alkyl”, by itself or as part of another substituent, means, unless otherwise stated, a straight or branched chain, or cyclic hydrocarbon radical, or combination thereof, which may be fully saturated, mono- or polyunsaturated and can include di- and multi-radicals, having the number of carbons designated (i.e., C₁₋₁₀ means one to ten carbons). Examples of saturated hydrocarbon radicals include groups such as methyl, ethyl, n-propyl, isopropyl, n-butyl, t-butyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl”, unless otherwise noted, is also meant to include those derivatives of alkyl defined in more detail below as “cycloalkyl” and “alkylene”. The term “alkylene” by itself or as part of another substituent means a divalent radical derived from alkane, as exemplified by —CH₂CH₂CH₂CH₂—. Typically, an alkyl group will have from 1 to 24 carbon atoms, with those having 10 or fewer carbon atoms being preferred. A “lower alkyl” or “lower alkylene” is a shorter chain alkyl or alkylene group, generally having eight or fewer carbon atoms, preferably four or fewer carbon atoms.

The term “alkoxy”, employed alone or in combination with other terms means, unless otherwise stated, an alkyl group, as defined above, connected to the remainder of the molecule via an oxygen atom, such as, for example, methoxy, ethoxy, 1-propoxy, 2-propoxy, and the higher homologs and isomers.

The term “heteroalkyl”, by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si, S, and wherein the nitrogen and sulfur atoms may optionally be oxidized and the nitrogen heteroatom may optionally be quarternized. The heteroatom(s) O, N and S may be placed at any position of the heteroalkyl group except for the position at which the alkyl group is attached to the remainder of the molecule. Examples include —CH₂—CH₂—O—CH₃, —CH₂—CH₂—NH—CH₃, —CH₂—CH₂—N(CH₃), —CH₂—S—CH₂—CH₃, —CH₂—CH₂—S(O)—CH₃, —CH₂—CH₂—S(O)₂—CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Also included in the term “heteroalkyl” are those radicals described in more detail below as “heteroalkylene” and “heterocycloalkyl.” The term “heteroalkylene by itself or as part of another substituent means a divalent radical derived from heteroalkyl, as exemplified by —CH₂—CH₂—S—CH₂—CH₂— and —CH₂—S—CH₂—CH₂—NH—CH₂—. For heteroalkylene groups, heteroatoms can also occupy either or both of the chain termini. Still further, for alkylene and heteroalkylene linking groups, as well as all other linking groups described herein, no specific orientation of the linking group is implied.

The terms “cycloalkyl” and “heterocycloalkyl”, by themselves or in combination with other terms, represent, unless otherwise stated, cyclic versions of “alkyl” and “heteroalky” respectively. The terms “cycloalkyl” and “heterocycloalkyl” are also meant to include bicyclic, tricyclic and polycyclic versions thereof. Additionally, for heterocycloalkyl, a heteroatom may occupy the position at which the heterocyclyl is attached to the remainder of the molecule. Examples of cycloalkyl include cyclopentyl, cyclohexyl, 1-cyclohexyl, 3-cyclohexyl, cyclopentyl, bicyclo[2.2.1]heptyl, bicyclo[2.2.2]octyl, adamantyl, and the like. Example of heterocycloalkyl include 1-(1,2,5,6-tetrahydropyridyl), 1-piperidinyl, 2-piperidinyl, 3-piperidinyl, 4-morpholinyl, 3-morpholinyl, 1,4-diazabicyclo[2.2.2]oct-2-yl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen” by themselves or as part of another substituent, mean, unless otherwise stated, a fluorine, chlorine, bromine or iodine atom. Additionally, terms such as “fluoroalkyl”, are meant to include monofluoroalkyl and polyfluoroalkyl.

The term “aryl”, employed alone or in combination with other terms (e.g., aryloxy, arylthioxy, arylalkyl) means, unless otherwise stated, an aromatic substituent which can be a single ring or multiple rings (up to three rings) which are fused together or linked covalently. The rings may each contain from zero to four heteroatoms selected from N, O and S, wherein the nitrogen and sulfur atoms are optionally oxidized, and the nitrogen atom(s) are optionally quaternized. The aryl groups that contain heteroatoms may be referred to as “heteroaryl” and can be attached to the remainder of the molecule through a carbon atom or a heteroatom. Non-limiting examples of aryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidinyl, 4-pyrimidinyl, 5-benzothiazolyl, purinyl, 2-benzimidazolyl, 5-indolyl, 1-isoquinolinyl, 5-isoquinolinyl, 2-quinoxalinyl, 5-quinoxalinyl, 3-quinolinyl, and 6-quinolinyl. Substituents for each of the above noted aryl ring systems are selected form the group of acceptable substituents described below.

The terms “arylalkyl” and “arylheteroalkyl” are meant to include those radicals in which an aryl group is attached to an aryl group (e.g., benzyl, phenethyl, pyridylmethyl and the like) or a heteroalkyl group (e.g. phenoxymethyl, 2-pyridyloxymethyl, 1-napthyloxy-3-propyl, and the like). The arylaklyl and arylheteroalkyl groups will typically contain from 1 to 3 aryl moieties attached to the alkyl or heteroalkyl portion by a covalent bond or by fusing the ring to, for example, a cycloalkyl or heterocycloalkyl group. For arylheteroalkyl groups, a heteroatom can occupy the position at which the group is attached to the remainder of the molecule. For example, the term “arylheteroalkyl” is meant to include benzyloxy, 2-phenylethoxy, phenethylamine, and the like.

Each of the above terms (e.g., “alkyl”, “heteroalkyl”, “aryl” etc) is meant to include both substituted and unsubstituted forms of the indicated radical. Preferable substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and hetercycloalkenyl) can be a variety of groups selected from: —OR, ═O, ═NR′, N—OR′,

NR′R″, —SR′, -halogen, —SiR′R″R′″, —OC(O)R′, —CO₂R′, —CONR′R″,

OC(O)NR′R′″, —NR″C(O)R′, —NR″C(O)NR′R′″, —NR″C(O)₂R′,

NHC(NH₂)═NH, —NR′C(NH₂)═NH, —NH—, C(NH₂)═NR′,

S(O)R′, —S(O)₂R′, —S(O)₂NR′R″, —CN and —NO₂ in a number ranging from zero to (2N+1), where N is the total number of carbon atoms in such a radical. Preferably, substituted alkyl groups will have from one to six independently selected substituents, more preferably from one to four independently selected substituents, most preferably from one to three independently selected substituents. In the substituents listed above, R′, R″ and R′″ each independently refer to hydrogen, unsubstituted (C₁₋₈)alkyl and heteroalkyl, unsubstituted aryl, aryl substituted with 1-3 halogens, unsubstituted alkyl, alkoxy or thioalkoxy groups or aryl-(C₁₋₄)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl.

Similarly, substituents for the aryl groups are varied and selected from: -halogen, —OR′, —OC(O)R′, —NR′R″, —SR′, —R′, —CN, —NO₂, —CO₂R′, —CONR′R″, —OC(O)NR′R″, —NR″C(O)R′, —NR″C(O)₂R′, —NR″C(O)NR′R′″, —NH—C(NH₂)═NH, —NR′C(NH₂)═N H, —NH—C(NH₂)═NR′, —SOR′, —S(O)₂R′, —S(O)₂NR′R″, —N₃, —CH(Ph)₂, perfluor(C₁₋₄)alkoxy, and perfluoro(C₁₋₄)alkyl, in a number ranging from zero to the total number of open valences on the aromatic ring system; and where R′ and R″ are independently selected from hydrogen, (C₁₋₈)alkyl and heteroalkyl, unsubstituted aryl, (unsubstituted aryl)-(C₁₋₄)alkyl, and (unsubstituted aryl)oxy-(C₁₋₄)alkyl. Preferably, substituted aryl groups will have from one to four independently selected substituents, more preferably from one to three independently selected substituents, most preferably from one to two independently selected substituents.

Two of the substituents on adjacent atoms of the aryl ring may optionally be replaced with a substituent of the formula -T-C(O)—(CH₂)_{q}—U—, wherein T and U are independently —NH—, —O—, CH₂ or a single bond, and q is an integer of from 0 to 2. Alternatively, two of the substituents on adjacent atoms of the aryl ring may optionally be replaced with a substituent of formula -A-(CH2)r-B—, wherein A and B are independently —CH₂—, —O—, —NH—, S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl ring may optionally be replaced with a substituent of the formula —(CH₂)ₛ—X—(CH₂)ₜ—, where s and t are integers of from 0 to 3, and X is —O—, —NR′—, —S—, —S(O)—, —S(O)₂—, or —S(O)₂NR′—. The substituent R′ in —NR′— and S(O)₂NR′— selected from hydrogen or unsubstituted (C₁₋₆)alkyl.

The term “heteroatom” is meant to include oxygen (O), nitrogen (N), sulfur (S) and silicon (Si).

One particularly preferred LXR agonist of the present invention is Compound Ia within the scope of compounds of formula (I). 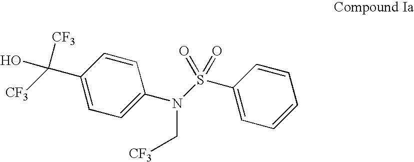
Compound Ia is described as Example 12 of WO 00/54759.

Compounds of formula (I) can be prepared using readily available starting materials or known intermediates. WO 00/54759 describes a number of possible synthetic routes for the production of such compounds, such as those depicted in scheme 1.

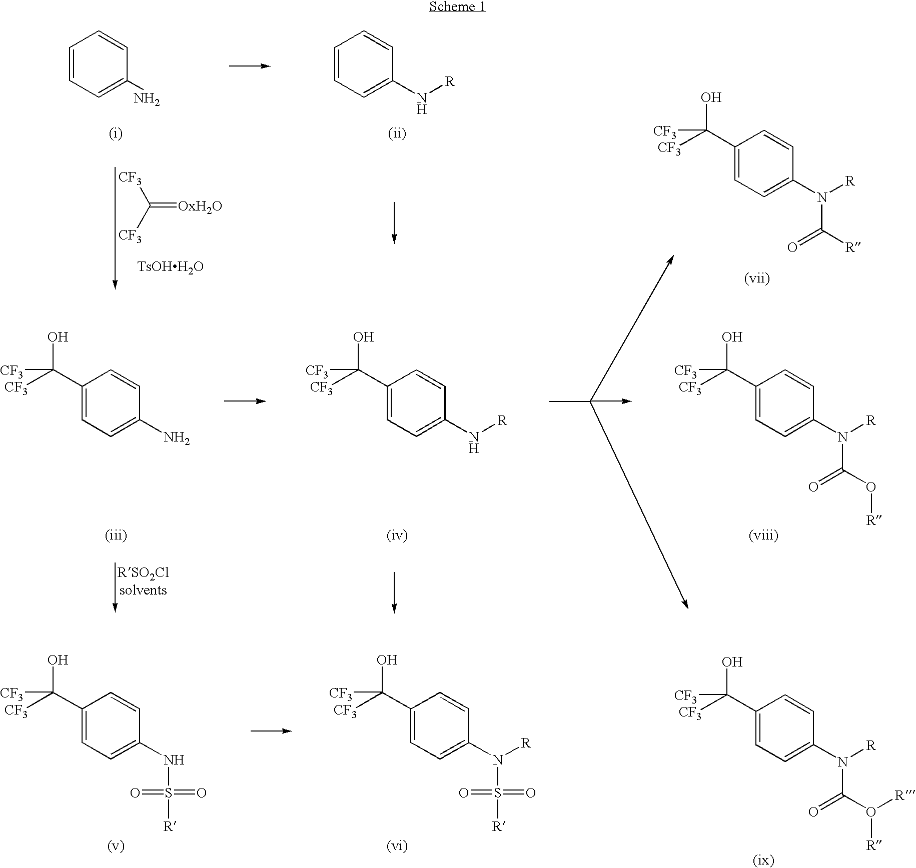

As shown in Scheme 1, aniline (i) (as representative of substituted anilines and other arylamines) can be alkylated, acylated or arylated (general addition of R group) to form (ii), or the aromatic ring can be derivatized with, for example, hexafluoroacetone to form (iii). Treatment of (iii) with an appropriate alkylating group, acylating group or arylating group provides (iv), which can be sulfonylated with, for example, an appropriate sulfonyl halide to form (vi). Alternatively, the aniline derivative can be sufonylated to form (v), which can then be alkylated or acylated to form compounds of formula (vi).

Other compounds of formula (I) can be formed by treating the substituted aniline (iv) (or iii), with reagents suitable for the formation of amides (vii), carbamates (viii) and ureas (ix). Various reagents are useful in the above scheme and can be found in, for example March, Advanced Organic Chemistry 4th ed. John Wiley & Sons, New York N.Y. (1992)

International Patent Application PCT/US01/27622 (SmithKline Beecham plc) discloses compounds of formula (II): 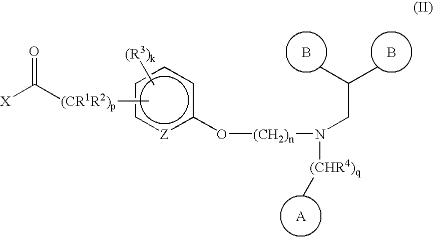
wherein:
X is OH or NH₂;
p is 0-6;
each R¹ and R² are the same or different and are each independently selected from the group consisting of H, C₁₋₈alkyl, C₁₋₈alkoxy and C₁₋₈thioalkyl;
Z is CH or N;
when Z is CH, k is 0-4;
when Z is N, k is 0-3;
each R³ is the same or different and is independently selected from the group consisting of halo, —OH, C₁₋₈alkyl, C₂₋₈alkenyl, C₁₋₈alkoxy, C₂₋₈alkenyloxy, —S(O)ₐR⁶, —NR⁷R⁸, —COR⁶, COOR⁶, R¹⁰COOR⁶, OR¹⁰COOR⁶, CONR⁷R⁸, —OC(O)R⁹, —R¹⁰NR⁷R⁸, —OR¹⁰NR⁷R⁸, 5-6 membered heterocycle, nitro, and cyano;
   a is 0, 1 or 2;
   R⁶ is selected from the group consisting of H, C₁₋₈alkyl, C₁₋₈alkoxy and C₂₋₈alkenyl;
   each R⁷ and R⁸ are the same or different and are each independently selected from the group consisting of H, C₁₋₈alkyl, C₂₋₈alkenyl, C₃₋₈alkynyl;
   R⁹ is selected from the group consisting of H, C₁₋₈alkyl and —NR⁷R⁸;
   R¹⁰ is C₁₋₈alkyl;
n is 2-8;
q is 0 or 1;
R⁴ is selected from the group consisting of H, C₁₋₈alkyl, C₁₋₈alkenyl, and alkenyloxy;
Ring A is selected from the group consisting of C₃₋₈cycloalkyl, aryl, 4-8 membered heterocycle, and 5-6 membered heteroaryl;
each ring B is the same or different and is independently selected from the group consisting of C₃₋₈cycloalkyl and aryl,
   as being useful as agonists of LXR and their use in pharmaceutical formulations to reverse cholesterol transport and treat atherosclerotic cardiovascular diseases and related diseases.

With respect to compounds of formula (II) the term “alkyl” refers to aliphatic straight or branched saturated hydrocarbon chains containing the specified number of carbon atoms. Examples of “alkyl” groups as used herein include but are not limited to methyl, ethyl, n-propyl, isopropyl, n-butyl, isobutyl, t-butyl, pentyl, hexyl, octyl and the like. The term “alkyl” also refers to substituted alkyl wherein the substituents are selected from the group consisting of halo, —OR⁷ and —SR⁷, where R⁷ is H or C₁₋₈alkyl. This definition of “alkyl” is also applicable to terms such as “thioalkyl” which incorporate the “alkyl” term. Thus, a “thioalkyl” as used herein refers to the group S—Ra where Ra is “alkyl” as defined.

The term “halo” refers to any halogen atom ie., fluorine, chlorine, bromine or iodine.

The term “alkenyl” refers to an aliphatic straight or branched unsaturated hydrocarbon chain containing at least one and up to three carbon-carbon double bonds. Examples of “alkenyl” groups as used herein include, but are not limited to, ethenyl and propenyl. The term “alkenyl” also refers to substituted alkenyl wherein the substituents are selected from the group consisting of halo, —OR⁷ and —SR⁷, where R⁷ is H or C₁₋₈alkyl.

The term “alkoxy” refers to a group O—Ra where Ra is “alkyl” as defined above.

The term “alkenyloxy” refers to a group O—Rb where Rb is “alkenyl” as defined above.

The term “cycloalkyl” refers to a non-aromatic carbocyclic ring having the specified number of carbon atoms and up to three carbon-carbon double bonds. “Cycloalkyl” includes by way of example cyclopropyl, cyclobutyl, cyclopentyl, cyclohexyl, cycloheptyl, cyclooctyl, cyclobutenyl, cyclopentenyl, cyclohexenyl and bicyclic cycloalkyl groups such as bicycloheptane and bicyclo(2.2.1)heptene. The term “cycloalkyl” also refers to substituted cycloalkyl wherein the ring bears one or more substituents selected from the group consisting of halo, —OH, C₁₋₈alkyl, C₂₋₈alkenyl, C₁₋₈alkoxy, C₂₋₈alkenyloxy, S(O)ₐR⁶, —NR⁷R⁸, —COR⁶, —COOR⁶, —R¹⁰COOR⁶, —OR¹⁰COOR⁶, —CONR⁷R⁸, —OC(O)R⁹, —R¹⁰NR⁷R⁸, —OR¹⁰NR⁷R⁸, nitro, and cyano, wherein a is 0, 1 or 2; R⁶ is selected from the group consisting of H, C₁₋₈alkyl, C₁₋₈alkoxy and C₂₋₈alkenyl; each R⁷ and R⁸ is the same or different and is independently selected from the group consisting of H, C₁₋₈alkyl, C₂₋₈alkenyl and C₃₋₈alkynyl; R⁹ is selected from the group consisting of H, C₁₋₈alkyl and —NR⁷R⁸; and R¹⁰ is C₁₋₈alkyl. As will be appreciated by those skilled in the art, the number of possible substituents on the cycloalkyl ring will depend upon the size of ring. In one preferred embodiment, the cycloalkyl is a cyclohexyl which may be substituted as described above.

The term “aryl” refers to aromatic groups selected from the group consisting of phenyl, 1-naphthyl and 2-naphthyl. The term “aryl” also refers to substituted aryl wherein the phenyl or naphthyl ring bears one or more substituents selected from the group consisting of halo, —OH, C₁₋₈alkyl, C₂₋₈alkenyl, C₁₋₈alkoxy, C₂₋₈alkenyloxy, S(O)ₐR⁶, —NR⁷R⁸, —COR⁶, —COOR⁶, —R¹⁰COOR⁶, —OR¹⁰COOR⁶, —CONR⁷R⁸, —OC(O)R⁹, —R¹⁰NR⁷R⁸, —OR¹⁰NR⁷R⁸, nitro, and cyano, wherein a is 0, 1 or 2; R⁶ is selected from the group consisting of H, C₁₋₈alkyl, C₁₋₈alkoxy and C₂₋₈alkenyl; each R⁷ and R⁸ is the same or different and is independently selected from the group consisting of H, C₁₋₈alkyl, C₂-₈alkenyl and C₃₋₈alkynyl; R⁹ is selected from the group consisting of H, C₁₋₈alkyl and —NR⁷R⁸; and R¹⁰ is C₁₋₈alkyl. As will be appreciated by those skilled in the art, the number of possible substituents on the aryl ring will depend upon the size of ring. For example, when the aryl ring is phenyl, the aryl ring may have up to 5 substituents selected from the foregoing list. One skilled in the art will readily be able to determine the maximum number of possible substituents for a 1-naphthyl or 2-naphthyl ring. A preferred aryl ring according to formula (II) is phenyl, which may be substituted as described above.

The term “heterocycle” refers to a monocyclic saturated or unsaturated non-aromatic carbocyclic rings and fused bicyclic non-aromatic carbocyclic rings, having the specified number of members in the ring and containing 1, 2 or 3 heteroatoms selected from N, O and S. Examples of particular heterocyclic groups include but are not limited to tetrahydrofuran, dihydropyran, tetrahydropyran, pyran, oxetane, thietane, 1,4-dioxane, 1,3-dioxane, 1,3-dioxalane, piperidine, piperazine, tetrahydropyrimidine, pyrrolidine, morpholine, thiomorpholine, thiazolidine, oxazolidine, tetrahydrothiopyran, tetrahydrothiophene, and the like. The term “heterocycle” also refers to substituted heterocycles wherein the heterocyclic ring bears one or more substituents selected from the group consisting of halo, —OH, C₁₋₈alkyl, C₂₋₈alkenyl, C₁₋₈alkoxy, C₂₋₈alkenyloxy, S(O)ₐR⁶, —NR⁷R⁸, —COR⁶, —COOR⁶, —R¹⁰COOR⁶, —OR¹⁰COOR⁶, —CONR⁷R⁸, —OC(O)R⁹, —R¹⁰NR⁷R⁸, —OR¹⁰NR⁷R⁸, nitro, and cyano, wherein a is 0, 1 or 2; R⁶ is selected from the group consisting of H, C₁₋₈alkyl, C₁₋₈alkoxy and C₂₋₈alkenyl; each R⁷ and R⁸ is the same or different and is independently selected from the group consisting of H, C₁₋₈alkyl, C₂₋₈alkenyl and C₃₋₈alkynyl; and R⁹ is selected from the group consisting of H, C₁₋₈alkyl and —NR⁷R⁸; and R¹⁰ is C₁₋₈alkyl. As will be appreciated by those skilled in the art, the number of possible substituents on the heterocyclic ring will depend upon the size of ring. There are no restrictions on the positions of the optional substituents in the heterocycles. Thus, the term encompasses rings having a substituent attached to the ring through a heteroatom. One skilled in the art will readily be able to determine the maximum number and locations of possible substituents for any given heterocycle. A preferred heterocycle according to the invention is piperidine, which may be substituted as described above.

The term “heteroaryl” refers to aromatic monocyclic heterocyclic rings and aromatic fused bicyclic rings having the specified number of members in the ring, having at least one aromatic ring and containing 1, 2 or 3 heteroatoms selected from N, O and S. Examples of particular heteroaryl groups include, but are not limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, and indazole. The term “heteroaryl” also refers to substituted heteroaryls wherein the heteroaryl ring bears one or more substituents selected from the group consisting of halo, —OH, C₁₋₈alkyl, C₂₋₈alkenyl, C₁₋₈alkoxy, C₂₋₈alkenyloxy, S(O)ₐR⁶, —NR⁷R⁸, —COR⁶, —COOR⁶, —R¹⁰COOR⁶, —OR¹⁰COOR⁶, —CONR⁷R⁸, —OC(O)R⁹, —R¹⁰NR⁷R⁸, —OR¹⁰NR⁷R⁸, nitro, and cyano, wherein a is 0, 1 or 2; R⁶ is selected from the group consisting of H, C₁₋₈alkyl, C₁₋₈alkoxy and C₂₋₈alkenyl; each R⁷ and R⁸ is the same or different and is independently selected from the group consisting of H, C₁₋₈alkyl, C₂₋₈alkenyl and C₃₋₈alkynyl; and R⁹ is selected from the group consisting of H, C₁₋₈alkyl and —NR⁷R⁸; and R¹⁰ is C₁₋₈alkyl. As will be appreciated by those skilled in the art, the number of possible substituents on the heteroaryl ring will depend upon the size of ring. There are no restrictions on the positions of the optional substituents in heteroaryls. Thus, the term encompasses rings having a substituent attached to the ring through a heteroatom. One skilled in the art will readily be able to determine the maximum number and locations of possible substituents for any given heteroaryl. A preferred heteroaryl according to the invention is pyridine, which may be substituted as described above.

The term “protecting group” refers to suitable protecting groups useful for the synthesis of compounds of formula (I) wherein X is OH. Suitable protecting groups are known to those skilled in the art and are described in Protecting Groups in Organic Synthesis, 3^{rd} Edition, Greene, T. W.; Wuts, P. G. M. Eds.; John Wiley & Sons: NY, 1999. Examples of preferred protecting groups include but are not limited to methyl, ethyl, benzyl, substituted benzyl, and tert-butyl. In one embodiment the protecting group is methyl.

Example 16 of PCT/US01/27622 (Smith Kline Beecham plc) has the following structure of formula (IIa) (hereinafter referred to as Compound IIa):

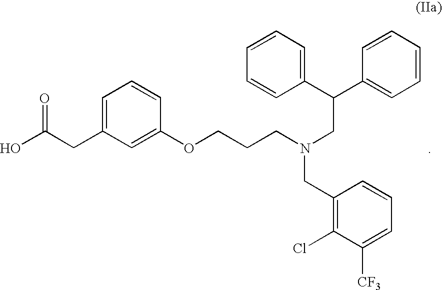

Compounds of formula (II) can be made according to any suitable method of organic chemistry. One method given in the specification is a solid phase synthesis process as depicted in Scheme 2.

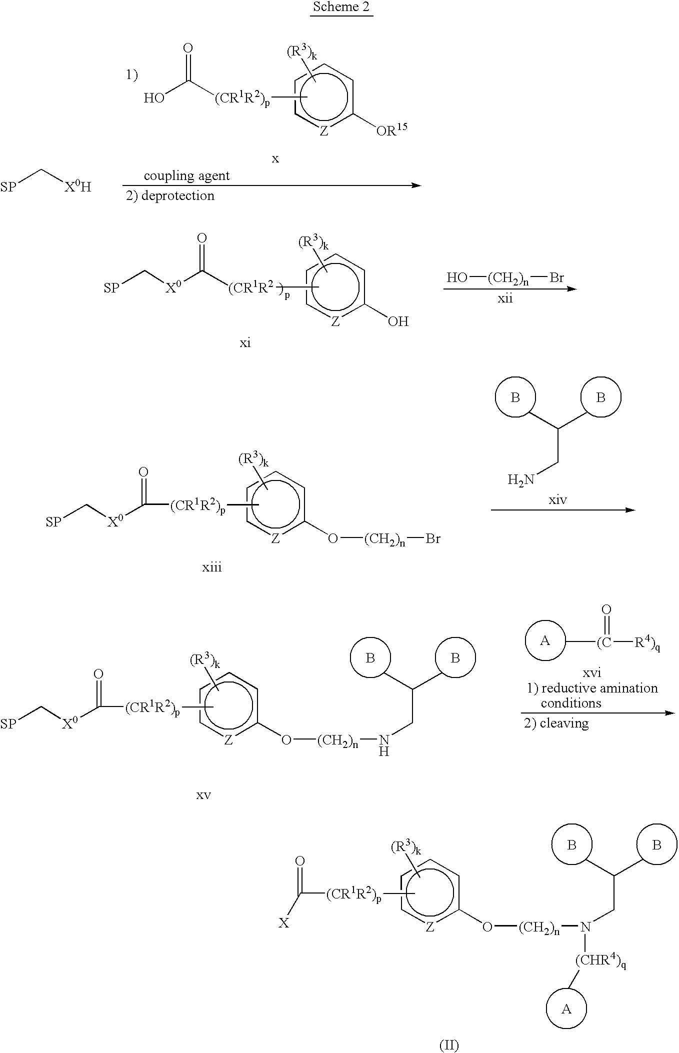

wherein X⁰ is —O— or —NH—, SP is solid phase, R¹⁵ is H or a protecting group, and all other variables are as defined above in connection with the description of compounds of formula (II).

In general, the reaction proceeds by a) reacting a solid phase-bound amine (where X in the compound of formula (II) is NH₂) or alcohol (where X in the compound of formula (II) is OH) with a compound of formula (x) and a coupling agent to produce a solid phase-bound compound of formula (xi); b) in the embodiment wherein R¹⁵ is a protecting group, deprotecting the solid phase bound compound to prepare the compound of formula (xi); c) alkylating the solid phase-bound compound of formula (xi) with an alcohol of formula (xii) to produce a solid phase-bound compound of formula (xiii); d) reacting the solid-phase-bound compound of formula (xiii) with a compound of formula (xiv) to produce the solid-phase bound compound of formula (xv); and e) reacting the solid phase-bound compound of formula (xv) with a compound of formula (xvi) under reductive amination conditions to produce the solid phase-bound compound of formula (II). The process may optionally further comprise the step of cleaving the solid phase-bound compound of formula (II) from the solid phase using conventional techniques such as treatment with mild acid.

Compounds of formula (II) are commercially available or can be prepared using conventional techniques such as those described in European Patent No. 303,742.

In one preferred embodiment, LXR agonists of the present invention relates to a compound of formula (II), and more preferably the compound of formula (IIa).

Compounds of formula (III) are described in U.S. Provisional application Ser. Nos. 09/368,427, 60/368,425 and 60/368,426, each filed Mar. 27, 2002: 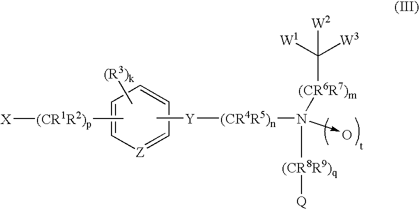
wherein:

X is selected from C₁-C₈ alkyl, halo, —OR¹⁰, —NR¹⁴R¹⁵, nitro, cyano, —COOR¹⁰, —COR¹³, —OCOR¹³, —CONR¹⁴R¹⁵, —N(R¹⁷)COR¹³, —N(R¹⁷)CONR¹⁴R¹⁵, —N(R¹⁷)COOR¹³, —SO₃H, —SO₂NR¹⁴R¹⁵, —C(═NR¹⁷)NR¹⁴R¹⁵, —N(R¹⁷)SO₂R¹⁶, and a 5 or 6-membered heterocyclic group;

or X and an adjacent R³, taken together with the atoms to which they are bonded, form an alkylenedioxy moiety;

Z is CH, CR³ or N, wherein when Z is CH or CR³, k is 0-4 and t is 0 or 1, and when Z is N, k is 0-3 and t is 0;

Y is selected from —O—, —S—, —N(R¹⁰)—, and —C(R⁴)(R⁵)—;

W¹ is selected from C₁-C₆ alkyl, C₃-C₈ cycloalkyl, aryl and Het, wherein said C₁-C₈ alkyl, C₃-C₈ cycloalkyl, Ar and Het are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, where said C₁-C₆ alkyl, is optionally unsubstituted or substituted by one or more halo substituents;

W² is selected from H, halo, C₁-C₆ alkyl, C₂-C₆ alkenyl, C₂-C₆ alkynyl, —C₀-C₆ alkyl-NR¹¹R¹², —C₀-C₆ alkyl-SR¹⁰, —C₀-C₆ alkyl-OR¹⁰, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-OCOR¹³, —C₀-C₆ alkyl-OCONR¹¹R¹², —C₀-C₆ alkyl-NR¹¹CONR¹¹R¹², —C₀-C₆ alkyl-NR¹¹COR¹³, —C₀-C₆ alkyl-Het, —C₀-C₆ alkyl-Ar and —C₀-C₆ alkyl-C₃-C₇ cycloalkyl, wherein said C₁-C₆ alkyl is optionally unsubstituted or substituted by one or more halo substituents, and wherein the C₃-C₇ cycloalkyl, Ar and Het moieties of said —C₀-C₆ alkyl-Het, —C₀-C₆ alkyl-Ar and —C₀-C₆ alkyl-C₃-C₇ cycloalkyl are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR¹⁰, —C₀-C₆ alkyl-CONR¹¹, R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR³, where said C₁-C₆ alkyl, is optionally unsubstituted or substituted by one or more halo substituents;

W³ is selected from the group consisting of: H, halo, C₁-C₆ alkyl, —C₀-C₆ alkyl-NR¹¹R¹², —C₀-C₆ alkyl-SR¹⁰, —C₀-C₆ alkyl-OR¹⁰, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-OCOR³, —C₀-C₆ alkyl-OCONR¹¹R¹², —C₀-C₆ alkyl-NR¹¹CONR¹¹R¹², —C₀-C₆ alkyl-NR¹¹COR³, —C₀-C₆ alkyl-Het, —C₁-C₆ alkyl-Ar and —C₁-C₆ alkyl-C₃-C₇ cycloalkyl, wherein said C₁-C₆ alkyl is optionally unsubstituted or substituted by one or more halo substituents;

Q is selected from C₃-C₈ cycloalkyl, Ar and Het; wherein said C₃-C₈ cycloalkyl, Ar and Het are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, where said C₁-C₆ alkyl is optionally unsubstituted or substituted by one or more halo substituents;

p is 0-8;

n is 2-8;

m is 0 or 1;

q is 0 or 1;

t is 0 or 1;

each R¹ and R² are independently selected from H, halo, C₁-C₆ alkyl, C₃-C₆ alkenyl, C₃-C₆ alkynyl, —C₀-C₆ alkyl-NR¹¹R¹², —C(—C₆ alkyl-OR¹⁰, —C₀-C₆ alkyl-SR¹⁰, —C₁-C₆ alkyl-Het, —C₁-C₆ alkyl-Ar and —C₁-C₆ alkyl-C₃-C₇ cycloalkyl, or R¹ and R² together with the carbon to which they are attached form a 3-5 membered carbocyclic or heterocyclic ring, wherein said heterocyclic ring contains one, or more heteroatoms selected from N, O, and S, where any of said C₁-C₆ alkyl is optionally unsubstituted or substituted by one or more halo substituents;

each R³ is the same or different and is independently selected from halo, cyano, nitro, C₁-C₆ alkyl, C₃-C₆ alkenyl, C₃-C₆ alkynyl, —C₀-C₆ alkyl-Ar, —C₀-C₆ alkyl-Het, —C₀-C₆ alkyl-C₃-C₇ cycloalkyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹³, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R², and —C₀-C₆ alkyl-NR¹¹COR³, wherein said C₃-C₆ alkyl is optionally unsubstituted or substituted by one or more halo substituents;

each R⁴ and R⁵ is independently selected from H, halo, C₁-C₆ alkyl, —C₀-C₆ alkyl-Het, —C₀-C₆ alkyl-Ar and —C₀-C₆ alkyl-C₃-C₇ cycloalkyl;

R⁶ and R⁷ are each independently selected from H, halo, C₁-C₆ alkyl, —C₀-C₆ alkyl-Het, —C₀-C₆ alkyl-Ar and —C₀-C₆ alkyl-C₃-C₇ cycloalkyl;

R⁸ and R⁹ are each independently selected from H, halo, C₁-C₆ alkyl, —C₀-C₆ alkyl-Het, —C₀-C₆ alkyl-Ar and —C₀-C₆ alkyl-C₃-C₇ cycloalklyl;

R¹⁰ is selected from H, C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl;

each R¹¹ and each R¹² are independently selected from H, C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl, or R¹¹ and R¹² together with the nitrogen to which they are attached form a 4-7 membered heterocyclic ring which optionally contains one or more additional heteroatoms selected from N, O, and S;

R¹³ is selected from C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl;

R¹⁴ and R¹⁵ are each independently selected from H, C₁-C₆ alkyl, C₃-C₆ alkenyl, C₃-C₆ alkynyl, —C₀-C₆ alkyl-Ar, —C₀-C₆ alkyl-Het, —C₀-C₆ alkyl-C₃-C₇ cycloalkyl, —C₀-C₆ alkyl-O—Ar, —C₀-C₆ alkyl-O-Het, —C₀-C₆ alkyl-O—C₃-C₇ cycloalkyl, —C₀-C₆ alkyl-S(O)ₓ—C₁-C₆ alkyl, —C₀-C₆ alkyl-S(O)ₓ—Ar, —C₀-C₆ alkyl-S(O)ₓ-Het, —C₀-C₆ alkyl-S(O), —C₃-C₇ cycloalkyl, —C₀-C₆ alkyl-NH-Het, —C₀-C₆ alkyl-NH—C₃-C₇ cycloalkyl, —C₀-C₆ alkyl-N(C₁-C₄ alkyl)-Ar, —C₀-C₆ alkyl-N(C₁-C₄ alkyl)-Het, —C₀-C₆ alkyl-N(C₁-C₄ alkyl)-C₃-C₇ cycloalkyl, —C₀-C₆ alkyl-Ar, —C₀-C₆ alkyl-Het and —C₀-C₆ alkyl-C₃-C₇ cycloalkyl, where x is 0, 1 or 2, or R¹⁴ and R¹⁵, together with the nitrogen to which they are attached, form a 4-7 membered heterocyclic ring which optionally contains one or more additional heteroatoms selected from N, O, and S, wherein said C₁-C₆ alkyl is optionally substituted by one or more of the substituents independently selected from the group halo, —OH, —SH, —NH₂, —NH(unsubstituted C₁-C₆ alkyl), —N(unsubstituted C₁-C₆ alkyl)(unsubstituted C₁-C₆ alkyl), unsubstituted —OC₁-C₆ alkyl, —CO₂H, —CO₂(unsubstituted C₁-C₆ alkyl), —CONH₂, —CONH(unsubstituted C₁-C₆ alkyl), —CON(unsubstituted C₁-C₆ alkyl)(unsubstituted C₁-C₆ alkyl), —SO₃H, —SO₂NH₂, —SO₂NH(unsubstituted C₁-C₆ alkyl) and —SO₂N(unsubstituted C₁-C₆ alkyl)(unsubstituted C₁-C₆ alkyl);

R¹⁶ is C₁-C₆ alkyl, —C₀-C₆ alkyl-Ar or —C₀-C₆ alkyl-Het; and

R¹⁷ is H, C₁-C₆ alkyl, —C₀-C₆ alkyl-Ar or —C₀-C₆ alkyl-Het.

Compounds of formula (IV) are described in U.S. Provisional Application No. 60/368,415, filed Mar. 27, 2002: 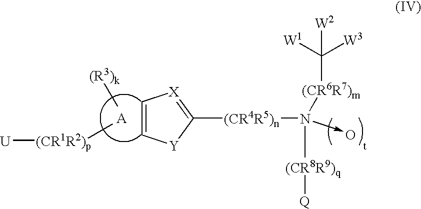
wherein:

X is CH or N;

Y is N(R¹⁰), O, or S, wherein t is 0 or 1 when Y is N(R¹⁰) or O, and t is 0 when Y is S;

U is selected from halo, —OR¹⁰, —NR¹⁴R¹⁵, nitro, cyano, —COOR¹⁰, —COR¹³, —OCOR³, —CONR¹⁴R¹⁵, —N(R¹⁴)COR¹³, —SO₃H, —SO₂NR¹⁴R¹⁵, —C(═NR¹⁷)NR¹⁴R¹⁵, —N(R¹⁴)SO₂R¹⁶, and a 5 or 6-membered heterocyclic group;

A is a phenyl fused ring moiety or a pyridyl fused ring moiety, wherein when A is a phenyl ring moiety, k is 0-3 and t is 0 or 1 and when A is a pyridyl ring moiety, k is 0-2 and t is 0;

W¹ is selected from C₃-C₈ cycloalkyl, aryl and Het, wherein said C₃-C₈ cycloalkyl, Ar and Het are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, where said C₁-C₆ alkyl, is optionally unsubstituted or substituted by one or more halo substituents;

W² is selected from H, halo, C₁-C₆ alkyl, C₂-C₆ alkenyl, C₂-C₆ alkynyl, —C₀-C₆ alkyl-NR¹¹R¹², —C₀-C₆ alkyl-SR¹⁰, —C₀-C₆ alkyl-OR¹⁰, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-OCOR¹³, —C₀-C₆ alkyl-OCONR¹¹R¹², —C₀-C₆ alkyl-NR¹¹CONR¹¹R¹², —C₀-C₆ alkyl-NR¹¹COR¹³, —C₀-C₆ alkyl-Het, —C₀-C₆ alkyl-Ar and —C₀-C₆ alkyl-C₃-C₇ cycloalkyl, wherein said C₁-C₆ alkyl is optionally unsubstituted or substituted by one or more halo substituents, and wherein the C₃-C₇ cycloalkyl, Ar and Het moieties of said —C₀-C₆ alkyl-Het, —C₀-C₆ alkyl-Ar and —C₀-C₆ alkyl-C₃-C₇ cycloalkyl are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, where said C₁-C₆ alkyl, is optionally unsubstituted or substituted by one or more halo substituents;

W³ is selected from the group consisting of: H, halo, C₁-C₆ alkyl, —C₀-C₆ alkyl-NR¹¹R¹², —C₀-C₆ alkyl-SR¹⁰, —C₀-C₆ alkyl-OR¹⁰, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-OCOR³, —C₀-C₆ alkyl-OCONR¹¹R¹², —C₀-C₆ alkyl-NR¹¹CONR¹¹R¹², —C₀-C₆ alkyl-NR¹¹COR¹³, —C₀-C₆ alkyl-Het, —C₁-C₆ alkyl-Ar and —C₁-C₆ alkyl-C₃-C₇ cycloalkyl, wherein said C₁-C₆ alkyl is optionally unsubstituted or substituted by one or more halo substituents;

Q is selected from C₃-C₈ cycloalkyl, Ar and Het; wherein said C₃-C₈ cycloalkyl, Ar and Het are optionally unsubstituted or substituted with one or more groups independently selected from halo, cyano, nitro, C₁-C₆ alkyl, C₃-C₆ alkenyl, C₃-C₆ alkynyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SO₂R¹³, —C₀-C₆ alkyl-OCOR¹³, —C₀-C₆ alkyl-OC(O)NR¹¹R¹², —C₀-C₆ alkyl-OC(O)OR¹³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, where said C₁-C₆ alkyl is optionally unsubstituted or substituted by one or more halo substituents;

p is 0-8;

n is 2-8;

m is 0 or 1;

q is 0 or 1;

t is 0 or 1;

each R¹ and R² are independently selected from H, halo, C₁-C₆ alkyl, C₃-C₆ alkenyl, C₃-C₆ alkynyl, —C₀-C₆ alkyl-NR¹¹R¹², —C₀-C₆ alkyl-OR¹⁰, —C₀-C₆ alkyl-SR¹⁰, —C₁-C₆ alkyl-Het, —C₁-C₆ alkyl-Ar and —C₁-C₆ alkyl-C₃-C₇ cycloalkyl, or R¹ and R² together with the carbon to which they are attached form a 3-5 membered carbocyclic or heterocyclic ring, wherein said heterocyclic ring contains one, or more heteroatoms selected from N, O, and S, where said C₁-C₆ alkyl is optionally unsubstituted or substituted by one or more halo substituents;

each R³ is the same or different and is independently selected from halo, cyano, nitro, C₁-C₆ alkyl, C₃-C₆ alkenyl, C₃-C₆ alkynyl, —C₀-C₆ alkyl-Ar, —C₀-C₆ alkyl-Het, —C₀-C₆ alkyl-C₃-C₇ cycloalkyl, —C₀-C₆ alkyl-CO₂R¹⁰, —C₀-C₆ alkyl-C(O)SR¹⁰, —C₀-C₆ alkyl-CONR¹¹R¹², —C₀-C₆ alkyl-COR¹³, —C₀-C₆ alkyl-NR¹¹R¹², —C₀-C₆ alkyl-SR¹⁰, —C₀-C₆ alkyl-OR¹⁰, —C₀-C₆ alkyl-SO₃H, —C₀-C₆ alkyl-SO₂NR¹¹R¹², —C₀-C₆ alkyl-SO₂R¹⁰, —C₀-C₆ alkyl-SOR¹³, —C₀-C₆ alkyl-OCOR¹³, —C₀-C₆ alkyl-OC(O)NR¹¹R¹², —C₀-C₆ alkyl-OC(O)OR³, —C₀-C₆ alkyl-NR¹¹C(O)OR¹³, —C₀-C₆ alkyl-NR¹¹C(O)NR¹¹R¹², and —C₀-C₆ alkyl-NR¹¹COR¹³, wherein said C₁-C₆ alkyl is optionally unsubstituted or substituted by one or more halo substituents;

each R⁴ and R⁵ is independently selected from H, halo, C₁-C₆ alkyl, —C₀-C₆ alkyl-Het, —C₀-C₆ alkyl-Ar and —C₀-C₆ alkyl-C₃-C₇ cycloalkyl;

R⁶ and R⁷ are each independently selected from H, halo, C₁-C₆ alkyl, —C₀-C₆ alkyl-Het, —C₀-C₆ alkyl-Ar and —C₀-C₆ alkyl-C₃-C₇ cycloalkyl;
R⁸ and R⁹ are each independently selected from H, halo, C₁-C₆ alkyl, —C₀-C₆ alkyl-Het, —C₀-C₆ alkyl-Ar and —C₀-C₆ alkyl-C₃-C₇ cycloalkyl;

R¹⁰ is selected from H, C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl;

each R¹¹ and each R¹² are independently selected from H, C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl, or R¹¹ and R¹² together with the nitrogen to which they are attached form a 4-7 membered heterocyclic ring which optionally contains one or more additional heteroatoms selected from N, O, and S;

R¹³ is selected from C₁-C₆ alkyl, C₃-C₆ alkenyl, C₃-C₆ alkynyl, —C₀-C₆ alkyl-Ar, —C₀-C₆ alkyl-Het and —C₀-C₆ alkyl-C₃-C₇ cycloalkyl;

R¹⁴ and R¹⁵ are each independently selected from H, C₁-C₆ alkyl, C₃-C₆ alkenyl, C₃-C₆ alkynyl, —C₀-C₆ alkyl-Ar, —C₀-C₆ alkyl-Het, —C₀-C₆ alkyl-C₃-C₇ cycloalkyl, —C₀-C₆ alkyl-O—Ar, —C₀-C₆ alkyl-O-Het, —C₀-C₆ alkyl-O—C₃-C₇ cycloalkyl, —C₀-C₆ alkyl-S(O)ₓ—C₁-C₆ alkyl, —C₀-C₆ alkyl-S(O)ₓ—Ar, —C₀-C₆ alkyl-S(O)ₓ-Het, —C₀-C₆ alkyl-S(O)ₓ—C₃-C₇ cycloalkyl, —C₀-C₆ alkyl-NH—Ar, —C₀-C₆ alkyl-NH-Het, —C₀-C₆ alkyl-NH—C₃-C₇ cycloalkyl, —C₀-C₆ alkyl-N(C₁-C₄ alkyl)-Ar, —C₀-C₆ alkyl-N(C₁-C₄ alkyl)-Het, —C₀-C₆ alkyl-N(C₁-C₄ alkyl)-C₃-C₇ cycloalkyl, —C₀-C₆ alkyl-Ar, —C₀-C₆ alkyl-Het and —C₀-C₆ alkyl-C₃-C₇ cycloalkyl, where x is 0, 1 or 2, or R¹⁴ and R¹⁵, together with the nitrogen to which they are attached, form a 4-7 membered heterocyclic ring which optionally contains one or more additional heteroatoms selected from N, O, and S, wherein said C₁-C₆ alkyl is optionally substituted by one or more of the substituents independently selected from the group halo, —OH, —SH, —NH₂, —NH(unsubstituted C₁-C₆ alkyl), —N(unsubstituted C₁-C₆ alkyl)(unsubstituted C₁-C₆ alkyl), unsubstituted —OC₁-C₆ alkyl, —CO₂H, —CO₂(unsubstituted C₁-C₆ alkyl), —CONH₂, —CONH(unsubstituted C₁-C₆ alkyl), —CON(unsubstituted C₁-C₆ alkyl)(unsubstituted C₁-C₆ alkyl), —SO₃H, —SO₂NH₂, —SO₂NH(unsubstituted C₁-C₆ alkyl) and —SO₂N(unsubstituted C₁-C₆ alkyl)(unsubstituted C₁-C₆ alkyl);

R¹⁶ is C₁-C₆ alkyl, —C₀-C₆ alkyl-Ar or —C₀-C₆ alkyl-Het; and

R¹⁷ is H, C₁-C₆ alkyl, —C₀-C₆ alkyl-Ar or —C₀-C₆ alkyl-Het.

Unless otherwise provided, each alkyl, alkoxy, alkenyl, alkynyl, cycloalkyl, aryl or Het (including any 3-5-membered, 4-7-membered or 5-7-membered carbocyclic or heterocyclic rings or ring moieties) in the compounds of formula (III) and (IV) is independently unsubstituted or substituted with one ore more substituents defined hereinbelow.

In the compounds of formula (IV), group A is defined as a phenyl or a pyridyl fused ring moiety and is exemplified by the following:

Group A fused ring moiety:

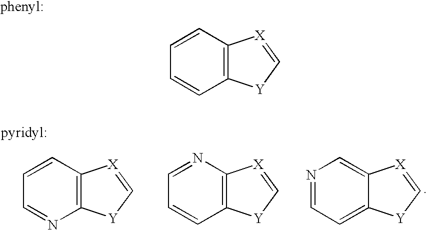

As used to define the compounds of formulas (III) or (IV), the term “alkyl” represents a straight- or branched-chain saturated hydrocarbon, containing 1 to 10 carbon atoms, unless otherwise provided, which may be unsubstituted or substituted by one or more of the substituents described below. Exemplary alkyls include, but are not limited to methyl (Me), ethyl (Et), n-propyl, isopropyl, n-butyl, isobutyl, t-butyl, n-pentyl, neopentyl and hexyl and structural isomers thereof. Any “alkyl” herein may be optionally substituted by one or more of the substituents independently selected from the group halo, —OH, —SH, —NH₂, —NH(unsubstituted C₁-C₆ alkyl), —N(unsubstituted C₁-C₆ alkyl)(unsubstituted C₁-C₆ alkyl), unsubstituted —OC₁-C₆ alkyl, and —CO₂H.

When combined with another substituent term as used to define the compounds of formulas (III) or (IV) (e.g., aryl or cycloalkyl as in -alkyl-Ar or -alkyl-cycloalkyl), the “alkyl” term therein refers to an alkylene moiety, that is, an unsubstituted divalent straight- or branched-chain saturated hydrocarbon moiety, containing 1 to 10 carbon atoms, unless otherwise provided. For example, the term “—C₀-C₆ alkyl-Ar”, where C is 1-6 is intended to mean the radical -alkyl-aryl (e.g., —CH₂-aryl or —CH(CH₃)-aryl) and is represented by the bonding arrangement present in a benzyl group. The term “C₀ alkyl” in a moiety, such as —C₀-C₆ alkyl-Ar or —O—(C₀-C₆ alkyl)-Ar, provides for no alkyl/alkylene group being present in the moiety. Thus, when C is zero, —C₀-C₆ alkyl-Ar is equivalent to —Ar and —O—(C₀-C₆ alkyl)-Ar is equivalent to —O—Ar.

As used to define the compounds of formulas (III) or (IV), the term “alkenyl” represents a straight- or branched-chain hydrocarbon, containing 2 to 10 carbon atoms, unless otherwise provided, and one or more carbon-carbon double bonds. Alkenyl groups may be unsubstituted or substituted by one or more of the substituents described below. Exemplary alkenyls include, but are not limited ethenyl, 1-propenyl, 2-propenyl, 1-butenyl, 2-butenyl, isobutenyl, butadienyl, pentenyl and hexenyl and structural isomers thereof. Both cis (Z) and trans (E) isomers of each double bond that may be present in the compounds of formula (III) or (IV) are included within the scope of this definition. Any “alkenyl” herein may be optionally substituted by one or more of the substituents independently selected from the group halo, —OH, —SH, —NH₂, —NH(unsubstituted C₁-C₆ alkyl), —N(unsubstituted C—C₆ alkyl)(unsubstituted C₁-C₆ alkyl), unsubstituted —OC₁-C₆ alkyl, and —CO₂H.

As used to define the compounds of formulas (III) or (IV), the term “alkynyl” represents a straight- or branched-chain hydrocarbon, containing 2 to 10 carbon atoms, unless otherwise provided, and one or more carbon-carbon triple bonds and, optionally, one or more carbon-carbon double bonds. Both cis (Z) and trans (E) isomers of each double bond that may be present in the compounds of formula (III) or (IV) are included within the scope of this definition. Exemplary alkynyls include, but are not limited ethynyl, propynyl (propargyl, isopropynyl), 1-butynyl, 2-butynyl, 3-butynyl, pentynyl and hexynyl and structural isomers thereof. Any “alkynyl” herein may be optionally substituted by one or more of the substituents independently selected from the group halo, —OH, —SH, —NH₂, —NH(unsubstituted C—C₆ alkyl), —N(unsubstituted C₁-C₆ alkyl)(unsubstituted C₁-C₆ alkyl), unsubstituted —OC—C₆ alkyl, and —CO₂H.

As used to define the compounds of formulas (III) or (IV), when an alkenyl or alkynyl group is a substituent on an oxygen, nitrogen or sulfur atom (e.g., as in oxy (—OR), thio (—SR), ester (—CO₂R or —C(O)SR), amino (—NRR) or amido (—CONRR) moieties and the like), it is understood that a double or triple bond of the alkenyl or alkynyl group is not located on carbons that are α,β to the oxygen, nitrogen or sulfur atom. Compounds containing ene-amino or enol-type moieties (—NR—CR═CR— or —O—CR═CR—) are not intended to be included within the scope of the definition of the compounds of formula (III) or (IV).

As used to define the compounds of formulas (III) or (IV), the term “cycloalkyl” represents a non-aromatic monocyclic, bicyclic, or tricyclic hydrocarbon containing from 3 to 10 carbon atoms which may be unsubstituted or substituted by one or more of the substituents described below and may be saturated or partially unsaturated. Exemplary cycloalkyls include monocyclic rings having from 3-7, preferably 3-6, carbon atoms, such as cyclopropyl, cyclobutyl, cyclopentyl, cyclopentenyl, cyclopentadienyl, cyclohexyl, cyclohexenyl and cycloheptyl. Any “cycloalkyl” herein may be optionally substituted by one or more of the substituents independently selected from the group halo, cyano, C₁-C₆ alkyl (which specifically includes C₁-C₆ haloalkyl, —C₀-C₆ alkyl-OH, —C₀-C₆ alkyl-SH and —C₀-C₆ alkyl-NR′R″), C₃-C₆ alkenyl, oxo, —OC₁-C₆alkyl, —OC₁-C₆ alkenyl, —C₀-C₆ alkyl-COR′, —C₀-C₆ alkyl-CO₂R′, —C₀-C₆ alkyl-CONR′R″, —OC₀-C₆ alkyl-CO₂H, —OC₂-C₆ alkyl-NR′R″, and —C₀-C₆ alkyl-SO₂NR′R″, wherein each R′ and R″ are independently selected from H or unsubstituted C₁-C₆ alkyl.

As used to define the compounds of formulas (III) or (IV), the terms “Ar” or “aryl” is used interchangeably at all occurrences mean a substituted or unsubstituted carbocyclic aromatic group, which may be optionally fused to another carbocyclic aromatic group moiety or to a cycloalkyl group moiety, which may be optionally substituted or unsubstituted. Examples of suitable Ar or aryl groups include phenyl, naphthyl indenyl, 1-oxo-1H-indenyl and tetrahydronaphthyl. Any “Ar”, “aryl” or “phenyl” herein may be optionally unsubstituted or substituted by one or more of the substituents independently selected from the group halo, cyano, C₁-C₆ alkyl (which specifically includes C₁-C₆ haloalkyl, —C₀-C₆ alkyl-OH, —C₀-C₆ alkyl-SH and —C₀-C₆ alkyl-NR′R″), C₃-C₆ alkenyl, —OC₁-C₆alkyl, —OC₁-C₆ alkenyl, —C₀-C₆ alkyl-COR′, —C₀-C₆ alkyl-CO₂R′, —C₀-C₆ alkyl-CONR′R″, —OC₀-C₆ alkyl-CO₂H, —OC₂-C₆ alkyl-NR′R″, —C₀-C₆ alkyl-C(═NR′)NR′R″, and —C₀-C₆ alkyl-SO₂NR′R″, wherein each R′ and R″ are independently selected from H or unsubstituted C₁-C₆ alkyl.

As used to define the compounds of formulas (III) or (IV), the term “Het” means a stable 5 to 7-membered monocyclic, a stable 7- to 10-membered bicyclic, or a stable 11- to 18-membered tricyclic heterocyclic ring group, all of which are saturated, unsaturated or aromatic, and consist of carbon atoms and from one to three heteroatoms selected from the group consisting of N, O and S, and which includes bicyclic and tricyclic rings containing one or more fused cycloalkyl, aryl (e.g., phenyl) or heteroaryl (aromatic Het) ring moieties. As used herein the term “Het” is also intended to encompass heterocyclic groups containing nitrogen and/or sulfur where the nitrogen or sulfur heteroatoms are optionally oxidized or the nitrogen heteroatom is optionally quaternized. The heterocyclic group may be attached at any heteroatom or carbon atom that results in the creation of a stable structure. Any “Het” herein may be optionally unsubstituted or substituted by one or more of the substituents independently selected from the group halo, cyano, C₁-C₆ alkyl (which specifically includes C₁-C₆ haloalkyl, —C₀-C₆ alkyl-OH, —C₀-C₆ alkyl-SH and —C₀-C₆ alkyl-NR′R″), C₃-C₆ alkenyl, oxo, —OC₁-C₆alkyl, —OC₁-C₆ alkenyl, —C₀-C₆ alkyl-COR′, —C₀-C₆ alkyl-CO₂R′, —C₀-C₆ alkyl-CONR′R″, —OC₀-C₆ alkyl-CO₂H, —OC₂-C₆ alkyl-NR′R″, —C₀-C₆ alkyl-C(═NR)NR′R″ and —C₀-C₆ alkyl-SO₂NR′R″, wherein each R′ and R″ are independently selected from H or unsubstituted C₁-C₆ alkyl.

Examples of such heterocyclic groups include, but are not limited to piperidinyl, piperazinyl, 2-oxopiperazinyl, 2-oxopiperidinyl, 2-oxopyrrolodinyl, 2-oxoazepinyl, azepanyl, pyrrolyl, 4-piperidonyl, pyrrolidinyl, pyrazolyl, pyrazolidinyl, imidazolyl, pyridinyl, pyrazinyl, oxazolidinyl, oxazolinyl, oxazolyl, isoxazolyl, morpholinyl, thiazolidinyl, thiazolinyl, thiazolyl, 1,3-benzodioxolyl (e.g., methylenedioxy-substituted phenyl), 1,4-benzodioxolyl, quinuclidinyl, indolyl, quinolinyl, isoquinolinyl, benzimidazolyl, benzopyranyl, benzoxazolyl, furyl, pyranyl, tetrahydrofuryl, tetrahydropyranyl, thienyl, benzoxazolyl, benzofuranyl, benzothienyl, dihydrobenzofuranyl, dihydrobenzothienyl, dihydroindolyl, tetrazolyl, thiamorpholinyl sulfoxide, thiamorpholinyl sulfone, and oxadiazolyl, as well as triazolyl, thiadiazolyl, oxadiazolyl, isoxazolyl, isothiazolyl, imidazolyl, pyridazinyl, pyrimidinyl and triazinyl which are available by routine chemical synthesis and are stable.

Examples of the 4-7 membered heterocyclic rings useful in the compounds of formula (III) or (IV), include, but are not limited to azetidinyl, piperidinyl, piperazinyl, 2-oxopiperazinyl, 2-oxopiperidinyl, 2-oxopyrrolodinyl, azepanyl, pyrrolyl, 4-piperidonyl, pyrrolidinyl, pyrazolyl, pyrazolidinyl, imidazolyl, pyridinyl, pyrazinyl, oxazolidinyl, oxazolinyl, oxazolyl, isoxazolyl, morpholinyl, thiazolidinyl, thiazolinyl, thiazolyl, furyl, pyranyl, tetrahydrofuryl, tetrahydropyranyl, thienyl, tetrazolyl, thiamorpholinyl sulfoxide, thiamorpholinyl sulfone, and oxadiazolyl, as well as triazolyl, thiadiazolyl, oxadiazolyl, isoxazolyl, isothiazolyl, imidazolyl, pyridazinyl, pyrimidinyl and triazinyl which are available by routine chemical synthesis and are stable. The 4-7 membered heterocyclic group may be optionally unsubstituted or substituted by one or more of the substituents independently selected from the group halo, cyano, C₁-C₆ alkyl (which specifically includes C₁-C₆ haloalkyl, —C₀-C₆ alkyl-OH, —C₀-C₆ alkyl-SH and —C₀-C₆ alkyl-NR′R″), C₃-C₆ alkenyl, oxo, —OC₁-C₆alkyl, —OC₁-C₆ alkenyl, —C₀-C₆ alkyl-COR′, —C₀-C₆ alkyl-CO₂R′, —C₀-C₆ alkyl-CONR′R″, —OC₀-C₆ alkyl-CO₂H, —OC₂-C₆ alkyl-NR′R″, —C₀-C₆ alkyl-C(═NR′)NR′R″ and —C₀-C₆ alkyl-SO₂NR′R″, wherein each R′ and R″ are independently selected from H or unsubstituted C₁-C₆ alkyl.

Examples of 5 or 6 membered heterocyclic groups include, but are not limited to piperidinyl, piperazinyl, 2-oxopiperazinyl, 2-oxopiperidinyl, 2-oxopyrrolodinyl, pyrrolyl, 4-piperidonyl, pyrrolidinyl, pyrazolyl, pyrazolidinyl, imidazolyl, pyridinyl, pyrazinyl, oxazolidinyl, oxazolinyl, oxazolyl, isoxazolyl, morpholinyl, thiazolidinyl, thiazolinyl, thiazolyl, furyl, pyranyl, tetrahydrofuryl, tetrahydropyranyl, thienyl, tetrazolyl, thiamorpholinyl sulfoxide, thiamorpholinyl sulfone, and oxadiazolyl, as well as triazolyl, thiadiazolyl, oxadiazolyl, isoxazolyl, isothiazolyl, imidazolyl, pyridazinyl, pyrimidinyl and triazinyl which are available by routine chemical synthesis and are stable. The 5-6 membered heterocyclic group may be attached at any heteroatom or carbon atom that results in the creation of a stable structure. The 5-6 membered heterocyclic group may be optionally unsubstituted or substituted by one or more of the substituents independently selected from the group halo, cyano, C₁-C₆ alkyl (which specifically includes C₁-C₆ haloalkyl, —C₀-C₆ alkyl-OH, —C₀-C₆ alkyl-SH and —C₀-C₆ alkyl-NR′R″), C₃-C₆ alkenyl, oxo, —OC₁-C₆alkyl, —OC₁-C₆ alkenyl, —C₀-C₆ alkyl-COR′, —C₀-C₆ alkyl-CO₂R′, —C₀-C₆ alkyl-CONR′R″, —OC₀-C₆ alkyl-CO₂H, —OC₂-C₆ akyl-NR′R″, —C₀-C₆ alkyl-C(═NR)NR′R″ and —C₀-C₆ alkyl-SO₂NR′R″, wherein each R′ and R″ are independently selected from H or unsubstituted C₁-C₆ alkyl.

In the compounds of formulas (III) and (IV), the terms “halogen” and “halo” represent chloro, fluoro, bromo or iodo substituents; “alkoxy” is intended to mean the radical —ORₐ, where Rₐ is an alkyl group, wherein alkyl is as defined above, provided that —O—C₁ alkyl may be optionally substituted by one or more of the substituents independently selected from the group halo and —CO₂H. (exemplary alkoxy groups include methoxy, ethoxy, propoxy, and the like); “phenoxy” is intended to mean the radical —ORₐᵣ, where Rₐᵣ is a phenyl group; “acetoxy” is intended to mean the radical —O—C(═O)-methyl; “benzoyloxy” is intended to mean the radical —O—C(═O)-phenyl; and “oxo” is intended to mean the keto diradical ═O, such as present on a pyrrolidin-2-one ring.

A method for the preparation of compounds of formula (III), comprises the steps of:

(a) reacting an alcohol having the formula: HY′—(CR⁴R⁵)ₙ-L, where Y′ is —O—, —S—, —NH or protected —NH and L is a leaving group, such as a halogen (iodide, bromide or chloride), sulfonate (tosylate, mesylate, triflate, etc.) or is a group that is converted to a leaving group (e.g., an alcohol), with an alcohol having the formula: 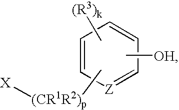
where X is a protected carboxylic acid moiety, to form a compound having the formula:

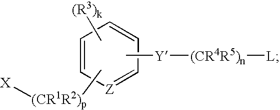

(b) reacting the compound formed in step (a) with a secondary amine having the formula 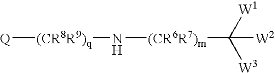
to form a compound having the formula:

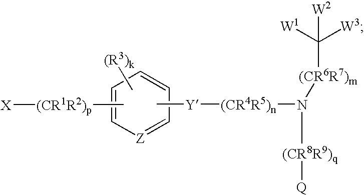

(c) converting the protected carboxylic acid moiety into a desired amide moiety; and

(d) optionally oxidizing the compound. formed in step (b) to the N-oxide thereof.

Another method for the preparation of compounds of formula (III), comprises the steps of:

(a) reacting an acetylene having the formula: R′O—(CR⁴R⁵)ₙ₋₁—C═C—H, where R′ is a hydroxyl protecting group, with a halogen-containing aromatic compound having the formula 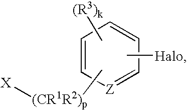
where X is a protected carboxylic acid moiety and Halo is bromo or iodo, in the presence of a catalyst to form a compound having the formula:

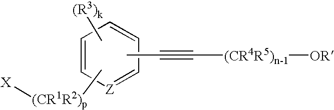

(b) reducing the compound formed in step (a) and converting the protected hydroxyl group into a leaving group, L, such as a halogen (iodide, bromide or chloride), sulfonate (tosylate, mesylate, triflate, etc.) or is a group that is converted to a leaving group (e.g., an alcohol), to form a compound having the formula:

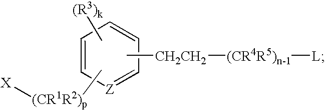

(c) reacting the compound formed in step (b) with an amine having the formula: 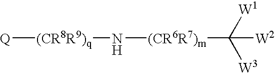
to form a compound having the formula:

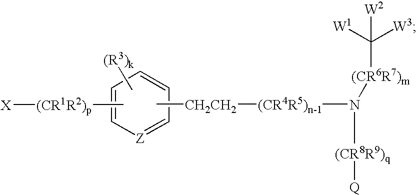

(d) converting the protected carboxylic acid moiety into a desired amide moiety; and

(e) optionally oxidizing the compound. formed in step (b) to the N-oxide thereof.

Another method for the preparation of compounds of formula (III), comprises the steps of:

(a) reacting an alcohol having the formula: L′-(CR⁴R⁵)ₙ-L, where L′ and L are leaving groups, which may be the same or different, such as a halogen (iodide, bromide or chloride), sulfonate (tosylate, mesylate, triflate, etc.) or is a group that is converted to a leaving group (e.g., an alcohol), with a compound having the formula: 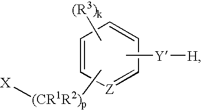
where Y′ is —O—, —S—, or —NH— and X is defined as above or a protected form thereof, to form a compound having the formula:

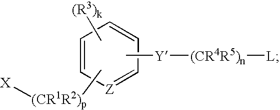

(b) reacting the compound formed in step (a) with a secondary amine having the formula 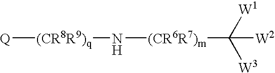
to form a compound having the formula:

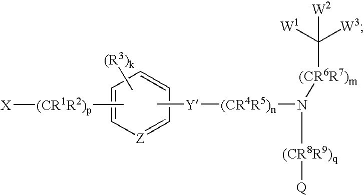

(c) removing any protecting groups; and

(d) optionally oxidizing the compound formed in step (b) or (c) to the N-oxide thereof.

Another method for the preparation of compounds of formula (III), comprises the steps of:

(a) reacting a compound having the formula: 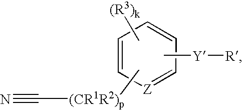
where Y′ is —O—, —S—, or —NH— and R′ is a suitable protecting group for —OH, —SH, or —NH₂, with a hydrazide or azide to form a heterocyclic-containing compound having the formula:

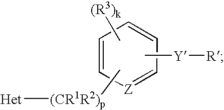

(b) optionally protecting the NH moiety of the heterocyclic group with a protecting group, and removing the R′ protecting group;

(c) reacting the compound formed in step (b) with a compound having the formula: L′-(CR⁴R⁵)ₙ-L, where L′ and L are leaving groups, which may be the same or different, such as a halogen (iodide, bromide or chloride), sulfonate (tosylate, mesylate, triflate, etc.) or is a group that is converted to a leaving group (e.g., an alcohol), to form a compound having the formula: 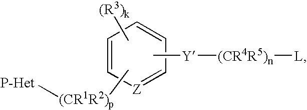
where P is an optional protecting group or H;

(d) reacting the compound formed in step (c) with an amine having the formula: 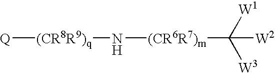
to form a compound having the structure:

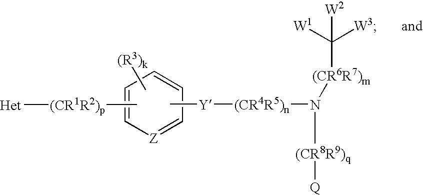

(e) removing any protecting groups.

Another method for the preparation of compounds of formula (I), comprises the steps of:

(a) reacting an acetylene having the formula: R′O—(CR⁴R⁵)ₙ₋₁—C═C—H, where R′ is a hydroxyl protecting group, with a halogen-containing aromatic compound having the formula 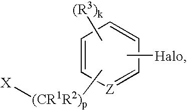
where Halo is bromo or iodo, in the presence of a catalyst to form a compound having the formula:

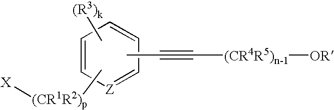

(b) reducing the compound formed in step (a) and converting the protected hydroxyl group into a leaving group, L, such as a halogen (iodide, bromide or chloride), sulfonate (tosylate, mesylate, triflate, etc.) or is a group that is converted to a leaving group (e.g., an alcohol) to form a compound having the formula:

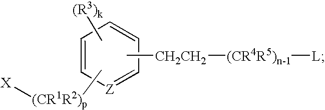

(c) reacting the compound formed in step (b) with an amine having the formula: 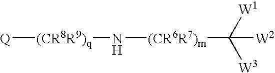
to form a compound having the formula:

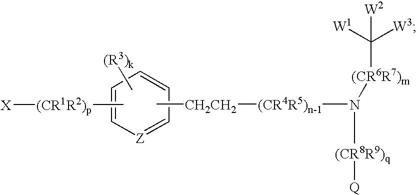

(d) removing any protecting groups; and

(e) optionally oxidizing the compound formed in step (c) or (d) to the N-oxide thereof.

Another method for the preparation of compounds of formula (E), comprises the steps of:

(a) reacting an alcohol having the formula: HO—(CR⁴R⁵)ₙ-L, where L is a leaving group, such as a halogen (iodide, bromide or chloride), sulfonate (tosylate, mesylate, triflate, etc.) or is a group that is converted to a leaving group (e.g., an alcohol) with a phenol having the formula: 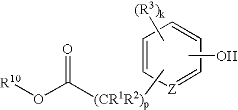
to form an aryl ether having the formula:

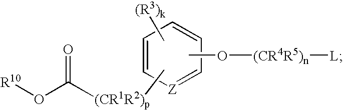

(b) reacting an amine having the formula 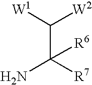
with and an aldehyde having the formula Q-CHO or a ketone to form a secondary amine having the formula:

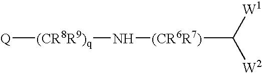

(c) reacting the ether formed in step (a) with the secondary amine formed in step (b) to form a compound of this invention having the formula:

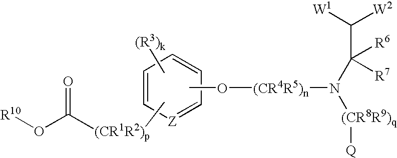

(d) when R¹⁰ is other than H, optionally converting the compound. formed in step (c) to the compound of this invention, wherein R¹⁰ is H.

Another method for the preparation of compounds of formula (III), comprises the steps of:

(a) reacting an alcohol having the formula: HO—(CR⁴R⁵)ₙ-L, where L is a leaving group, such as a halogen (iodide, bromide or chloride), sulfonate (tosylate, mesylate, triflate, etc.) or is a group that is converted to a leaving group (e.g., an alcohol), with an amine having the formula: 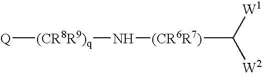
to form a tertiary amine having the formula:

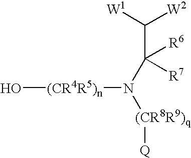

(b) reacting the tertiary amine formed in step (a) with a phenol having the formula: 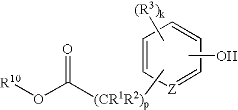
to form a compound of this invention having the formula:

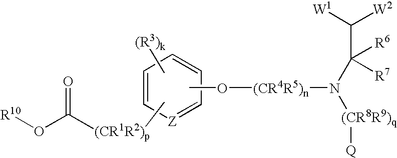

(c) when R¹⁰ is other than H, optionally converting the compound. formed in step (b) to the compound of this invention, wherein R¹⁰ is H.

Another method for the preparation of compounds of formula (III), comprises the steps of:

(a) reacting an alcohol having the formula: HO—(CR⁴R⁵)ₙ-L, where L is a leaving group, such as a halogen (iodide, bromide or chloride) or sulfonate (tosylate, mesylate, triflate, etc.), with a phenol having the formula: 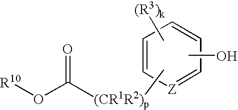
to form an ether-alcohol having the formula:

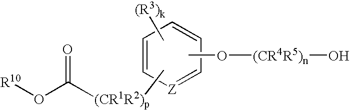

(b) converting alcohol moiety of the ether-alcohol formed in step (a) into L′, where L′ is a leaving group such as a halogen (iodide, bromide or chloride), sulfonate (tosylate, mesylate, triflate, etc.) or is a group that is converted to a leaving group (e.g., an alcohol) and treating the resulting compound with an amine having the formula: 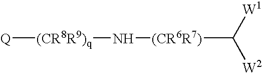
to form a compound of this invention having the formula:

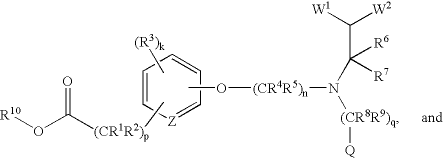

(c) when R¹⁰ is other than H, optionally converting the compound. formed in step (b) to the compound of this invention, wherein R¹⁰ is H.

The method for the preparation of compounds of formula (IV), comprises the steps of:

(a) coupling an acetylene having the formula: with a phenol having the formula: 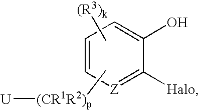
where Halo is a halogen selected from iodo or bromo, in the presence of a metal catalyst to form an aryl-alcohol having the formula:

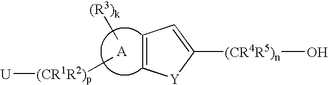

(b) converting alcohol moiety of the aryl-alcohol formed in step (a) into L′, where L′ is a leaving group such as a halogen (iodide, bromide or chloride), sulfonate (tosylate, mesylate, triflate, etc.) or is a group that is converted to a leaving group (e.g., an alcohol), and treating the resulting compound with an amine having the formula: 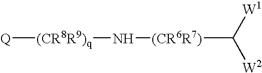
to form the compound of formula (IV);

(c) optionally converting the compound of formula (IV) from step (b) into another compound of formula (IV); and

(d) optionally oxidizing the compound. formed in step (c) to the N-oxide thereof

Alternatively, the compounds of formula (IV) may be prepared by

(a) coupling an acetylene having the formula: with a phenol having the formula: 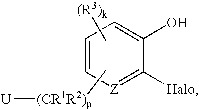
where Halo is a halogen selected from iodo or bromo, in the presence of a metal catalyst to form an aryl-alcohol having the formula:

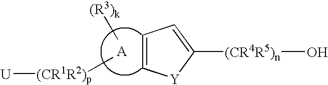

(b) converting alcohol moiety of the aryl-alcohol formed in step (a) into L′, where L′ is a leaving group such as a halogen (iodide, bromide or chloride) or a sulfonate (tosylate, mesylate, triflate, etc.) and treating the resulting compound with sodium azide, followed by hydrogenation in the presence of a palladium catalyst to form a primary amine having the formula:

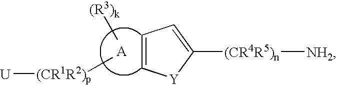

(c) treating the primary amine with a first aldehyde in the presence of a reducing agent, to form a secondary amine and treating the secondary amine with a second aldehyde in the presence of a reducing agent to form the compound of formula (IV);

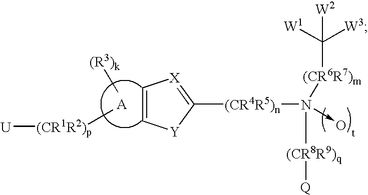

(d) optionally converting the compound of formula (IV) from step (b) into another compound of formula (IV); and

(e) optionally oxidizing the compound. formed in step (b) or (c) to the N-oxide thereof.

International Patent Applications WO 01/41704 (Merck & Co., Inc.) discloses compound of formula (V) 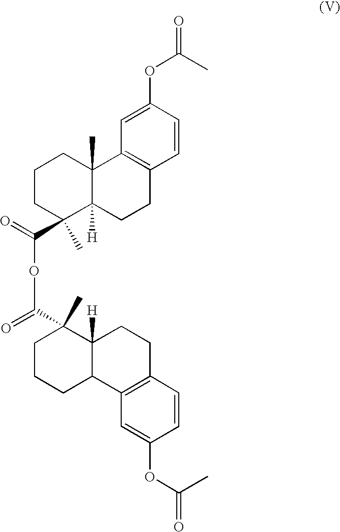
as being an agonist of LXR and its use in pharmaceutical formulations to prevent and treat atherosclerotic disease.

Other LXR agonists may be identified by assays such as those described in the above referenced patent applications, for example, the assays described in Examples 1 and 2 of PCT/US01/27622. Biotinylated LXRβ protein was incubated for 20-25 minutes at a concentration of 25 nM in assay buffer (50 mM KCl, 50 mM Tris-pH8, 0.1 mg/ml FAF-BSA, 10 mM DTT) with equimolar amounts of streptavidin-AlloPhycoCyanin (APC, Molecular Probes). At the same time, the biotinylated peptide comprising amino acids 675-699 of SRC-1 (CPSSHSSLTERHKILHRLLQEGSPS-CONH2) (SEQ ID NO: 5) at a concentration of 25 nM was incubated in assay buffer with a 12 molar amount of streptavidin-labelled Europium (Wallac) for 20-25 minutes. After the initial incubations are completed, a 10 molar excess (250 nM) of cold biotin was added to each of the solutions to block the unattached streptavidin reagents. After 20 min at room temp, the solutions were mixed yielding a concentration of 12.5 nM for the dye-labelled LXRβ protein and SRC-1 peptide.

80 μL of the protein/peptide mixture was added to each well of an assay plate containing 20 μL of test compound. The final volume in each well was 0.1 mL, and the concentration in the well for the dye-labelled protein and peptide was 10 nM. The final test compound concentrations were between 56 pM and 10 μM. The plates were incubated at room temp in the dark for 4-12 hours and then counted on a Wallac Victor fluorescent plate reader. In this assay 1 μM 24(S), 25-epoxycholesterol gave a reading of 20000 fluorescence units over a background reading of 10000 fluorescence units. The assay for LXRα was run according to the procedures described above using his-tagged LXRα ligand binding domain (amino acids 183-447 of Genbank accession number U22662, with the 14^{th} amino acid corrected to A from R).

Suitable pharmaceutically acceptable salts include salts of salts derived from appropriate acids, such as acid addition salts, or bases.

Suitable pharmaceutically acceptable salts include metal salts, such as for example aluminium, alkali metal salts such as lithium, sodium or potassium, alkaline earth metal salts such as calcium or magnesium and ammonium or substituted ammonium salts, for example those with lower alkylamines such as triethylamine, hydroxy alkylamines such as 2-hydroxyethylamine, bis-(2-hydroxyethyl)-amine or tri-(2-hydroxyethyl)-amine, cycloalkylamines such as bicyclohexylamine, or with procaine, dibenzylpiperidine, N-benzyl-b-phenethylamine, dehydroabietylamine, N,N′-bisdehydroabietylamine, glucamine, N-methylglucamine or bases of the pyridine type such as pyridine, collidine, quinine or quinoline.

Suitable acid addition salts include pharmaceutically acceptable inorganic salts such as the sulphate, nitrate, phosphate, borate, hydrochloride and hydrobromide and pharmaceutically acceptable organic acid addition salts such as acetate, tartrate, maleate, citrate, succinate, benzoate, ascorbate, methane-sulphonate, a-keto glutarate and a-glycerophosphate.

The LXR agonists referred to herein are conveniently prepared according to the methods disclosed in the above mentioned patent publications in which they are disclosed.

The salts and/or solvates of the LXR agonists may be prepared and isolated according to conventional procedures for example those disclosed in the, above mentioned, patent publications.

In the above mentioned method the LXR agonist, may be administered per se or, preferably, as a pharmaceutical composition/formulation also comprising a pharmaceutically acceptable carrier.

In the treatment of the invention, the LXR agonist mentioned herein is formulated and administered in accordance with the methods disclosed in the above mentioned patent applications and patents.

As used herein the term ‘pharmaceutically acceptable’ embraces compounds, compositions and ingredients for both human and veterinary use: for example the term ‘pharmaceutically acceptable salt’ also embraces a veterinarily acceptable salt.

Preferred “mammal” of the present invention is a human being.

The composition may, if desired, be in the form of a pack accompanied by written or printed instructions for use.

Usually the pharmaceutical compositions of the present invention will be adapted for oral administration, although compositions for administration by other routes, such as by injection, enema, colonoscopic infusion, infusion into the small bowel via an endoscope or intubation, and percutaneous absorption are also envisaged.

Particularly suitable compositions for oral administration are unit dosage forms such as tablets and capsules. Other fixed unit dosage forms, such as powders presented in sachets, may also be used.

In accordance with conventional pharmaceutical practice, the carrier may comprise a diluent, filler, disintegrant, wetting agent, lubricant, colourant, flavourant or other conventional adjuvant.

Typical carriers include, for example, microcrystalline cellulose, starch, sodium starch glycollate, polyvinylpyrrolidone, polyvinylpolypyrrolidone, magnesium stearate, sodium lauryl sulphate or sucrose.

The solid oral compositions may be prepared by conventional methods of blending, filling or tabletting. Repeated blending operations may be used to distribute the active agent throughout those compositions employing large quantities of fillers. Such operations are of course conventional in the art. The tablets may be coated according to methods well known in normal pharmaceutical practice, in particular with an enteric coating.

Oral liquid preparations may be in the form of, for example, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminum stearate gel, hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

For treating or preventing IBD, pharmaceutical composition (formulation) which delivers drug in colon is particularly preferred. For example, the oral formulations include prodrugs with enteric coatings. The prodrug formulation may require spontaneous or enzymatic transformation within the biological environment in order to release the drug. The release of the drug from the prodrug can be accomplished by formulation coated with pH sensitive polymer, hydrophilic or hydrophobic polymer along with enteric polymer, microbially degradable polymers (azo polymers) or polysaccharides. Various pharmaceutical approaches to colon targeted drug delivery system is well described by M. K. Chourasia and S. K. Jain in J Pharm Pharmaceut Sci 6(1):33-66, 2003.

For parenteral administration, fluid unit dosage forms are prepared utilizing the compound and a sterile vehicle, and, depending on the concentration used, can be either suspended or dissolved in the vehicle. In preparing solutions the compound can be dissolved in water for injection and filter sterilized before filling into a suitable vial or ampoule and sealing. Advantageously, adjuvants such as a local anaesthetic, a preservative and buffering agents can be dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

Compositions may contain from 0.1% to 99% by weight, preferably from 10-60% by weight, of the active material, depending upon the method of administration.

The compositions are formulated according to conventional methods, such as those disclosed in standard reference texts, for example the British and US Pharmacopoeias, Remington's Pharmaceutical Sciences (Mack Publishing Co.), Martindale The Extra Pharmacopoeia (London, The Pharmaceutical Press) and Harry's Cosmeticology (Leonard Hill Books).

Typically, a therapeutically effective amount of LXR agonist of the present invention for preventing or treating IBD will depend upon a number of factors including, for example, the age and weight of the mammal, the precise condition requiring treatment, the severity of the condition, the nature of the formulation, and the route of administration. Ultimately, the therapeutically effective amount will be at the discretion of the attendant physician or veterinarian.

Typically, the LXR agonist agent will be given in the range of 0.1 to 100 mg/kg body weight of recipient (mammal) per day and more usually in the range of 1 to 30 mg/kg body weight per day. Acceptable daily dosages of the LXR agonist for preventing/treating IBD may be from about 0.1 to about 1000 mg/day, and preferably from about 0.2 to about 100 mg/day.

The following Examples are intended for illustration only and are not intended to limit the scope of the invention in any way; the present invention being defined by the appended claims.

### EXAMPLES

### Example 1

### 2-(3-{3-[[2-Chloro-3-(trifluoromethyl)benzyl](2,2-diphenylethyl)amino]propoxy}-phenyl)acetic acid (IIa)

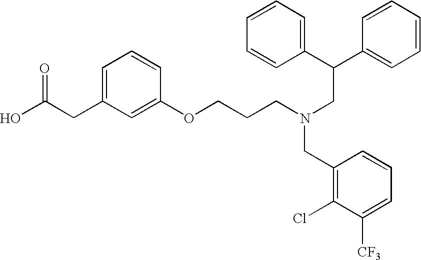

Argogel-MB-OH (6.0 g, 2.40 mmol, Argonaut Technologies) was treated with a solution of (3-{[tert-butyl(dimethyl)silyl]oxy}phenyl)acetic acid (5.40 g, 19.2 mmol, Eur. Pat. Appl. (1987) Application: EP 87-303742 19870428) in 50 mL of anhydrous dichloromethane followed by dicyclohexylcarbodiimide (4.16 g, 19.2 mmol) and 4-dimethylaminopyridine (2.50 g, 19.2 mmol). After rotating at room temperature for 15 hours, the resin was filtered, washed sequentially with dichloromethane (2×25 mL), dimethylformamide (2×25 mL), dichloromethane (3×25 mL), methanol (3×25 mL), dichloromethane (3×25 mL) and diethyl ether (2×25 mL). After drying under house vacuum overnight at 40° C., the resin was treated with 1.0 M tetrabutylammonium fluoride (24 mL, 23.4 mmol) in tetrahydrofuran, and the mixture was rotated for 4 hours. The resin was filtered, washed sequentially with dichloromethane (2×25 mL), dimethylformamide (2×25 mL), dichloromethane (3×25 mL), methanol (3×25 mL), and dichloromethane (3×25 mL) to give the deprotected phenol. The dry resin was treated with 90 mL of anhydrous toluene followed by triphenylphosphine (15.8 g, 60.0 mmol) and 3-bromo-1-propanol (8.4 g, 60.0 mmol). Upon cooling to 0° C., diisopropyl azodicarboxylate (12.1 g, 60.0 mmol) in 20 mL of anhydrous toluene was added in a dropwise fashion. The reaction was allowed to warm to room temperature and stirred for 15 hours. The resin was filtered, washed sequentially with dichloromethane (2×50 mL), dimethylformamide (2×50 mL), dichloromethane (3×50 mL), methanol (2×50 mL) and dichloromethane (3×50 mL), and dried under house vacuum. The bromide functionalized resin was treated with a solution of diphenethylamine (25.0 g, 127 mmol) in 60 mL of anhydrous dimethylsulfoxide, and the reaction was rotated for 15 hours. The resin was filtered, washed sequentially with dichloromethane (2×50 mL), dimethylformamide (2×50 mL), dichloromethane (3×50 mL), methanol (3×50 mL) and dichloromethane (3×50 mL), and dried under house vacuum at 40° C. The secondary amine resin (5.75 g, 2.0 mmol) was treated with a solution of 2-chloro-3-trifluoromethylbenzaldehyde (8.32 g, 40.0 mmol) in 80 mL of 8% acetic acid in dimethylformamide. Solid sodium triacetoxyborohydride (8.5 g, 40.0 mmol) was added, and the reaction was rotated for 15 hours. The resin was filtered, washed sequentially with dichloromethane (2×50 mL), dimethylformamide (2×50 mL), dichloromethane (3×50 mL), methanol (3×50 mL) and dichloromethane (3×50 mL), and dried under house vacuum overnight at 50° C. The resin-bound product was treated with 30 mL of trifluoroacetic acid/dichloromethane (15/85) for 15 minutes, and the filtrate was collected. The cleavage procedure was repeated again, and the combined filtrates were concentrated under reduced pressure. The crude product was purified by preparative thin layer chromatography (silica gel, 1 mm plates, Merck 20×20 cm silica gel 60 F₂₅₄) eluting with methanol:dichloromethane (3:97) to give 7.0 mg of the title compound (5% yield based on theoretical loading of secondary amine resin) of a viscous oil: ¹H NMR (CDCl₃, 400 MHz) δ 7.42 (d, 1H, J=7.6), 7.23-7.10 (m, 12H), 6.85 (t, 2H, J=8.1), 6.63 (s, 1H), 6.61 (s, 1H), 4.11 (t, 1H, J=7.8), 3.75 (s, 2H), 3.63 (t, 2H, J=6.0), 3.59 (s, 2H), 2.12 (d, 2H, J=7.8), 2.67 (t, 2H, J-6.6), 1.81 (tt, 2H, J=6.2); MS (ESP+) m/e 582(MH⁺); TLC (EtOAc:hexanes/1:1) R_{f}=0.58.

### Example 2

Induction of Colitis

Female, 10-week-old BALB/c mice (Charles River Japan) were used in this study. Colitis was induced by providing drinking water containing 3% dextran sulfate sodium (DSS, ICN Biomedicals Inc., M.W.=36,000-50,000) for 5 days. The administration of DSS was discontinued on day 5, and mice were given tap water alone for 7 days until on day 12.

Evaluation of Colitis

The disease activity index (DAI) was determined in all animals, by scoring body weight, stool consistency and rectal bleeding as described by Murthy, S. N. S. (Digestive Diseases and Sciences, 38(9) p. 1722-1734(1993)). The method of scoring is shown in Table 1. Severity of colitis was evaluated by area under the curve (AUC) calculated based on DAI curve ranged from day 3 to day 7 (AUC (3-7 day)), from day 7 to day 10 (AUC (7-10 day)), from day 10 to day 12 (AUC (10-12 day)) and from day 0 to day 12 (AUC (0-12 day)).

TABLE 1
Criteria for scoring

| | | Stool | Occult blood |
|---|---|---|---|
| Score | Weight loss (%) | consistency | or gross bleeding |
| 0 | None | Normal | Negative |
| 1 | 1-5 | Loose stool | Negative |
| 2 | 5-10 | Severe | Hemoccult positive |
| | | loose stool | |
| 3 | 10-15 | Diarrhea | Hemoccult strong |
| | | | positive |
| 4 | >15 | Severe diarrhea | Gross bleeding |

DAI = (combined score of weight loss, stool consistency and bleeding)/3.

Experimental Design

Ten mice were used in each group. Compound IIa and Compound Ia were suspended in 0.5% methylcellulose (MC) solution. Compound IIa 3 or 10 or 30 mg/kg or its vehicle (0.5% MC solution) was administered orally twice a day for 12 days from day 0. Compound Ia at 50 mg/kg was administered orally once a day for 12 days from day 0. The experimental groups were set up as follows:

Control*

3% DSS+vehicle (0.5% MC solution)
   3% DSS+Compound IIa (3 mg/kg)
   3% DSS+Compound IIa (10 mg/kg)
   3% DSS+Compound IIa (30 mg/kg)
   3% DSS+Compound Ia (50 mg/kg)
   * Mice which received tap water without DSS.

### Results

Effects of Compound IIa and Compound Ia on DSS-Induced Colitis

Effects of Compound IIa and Compound Ia on DSS-colitis were shown in Table 2. Compound IIa (3, 10 and 30 mg/kg, p.o., b.i.d.) suppressed the severity of DSS-induced colitis as expressed by a significantly lower AUC (3 mg/kg: AUC (3-7 day), 10 mg/kg: AUC (3-7 day), AUC (7-10 day) and AUC (0-12 day), 30 mg/kg: AUC (3-7 day) and AUC (0-12 day)) compared with vehicle-treated DSS-fed mice. Compound Ia (50 mg/kg, p.o., q.d.) inhibited the severity of DSS-induced colitis as expressed by a significant lower AUC (3-7 day) and AUC (0-12 day) compared with vehicle-treated DSS-fed mice.

TABLE 2
Evaluation of colitis by AUC

| Groups | n | AUC (3-7 day) | AUC (7-10 day) | AUC (10-12 day) | AUC (0-12 day) |
|---|---|---|---|---|---|
| Control | 10 | 0.73 +/− 0.23 | 0.45 +/− 0.19 | 0.35 +/− 0.16 | 1.98 +/− 0.63 |
| DSS + vehicle | 10 | 6.87 +/− 0.58 | 6.35 +/− 0.60 | 3.20 +/− 0.41 | 17.97 +/− 1.54 |
| DSS + IIa (3 mg/kg) | 10 | 4.93 +/− 0.38** | 5.05 +/− 0.75 | 2.73 +/− 0.55 | 14.02 +/− 1.56 |
| Inhibition (%) | | (28.2) | (20.5) | (14.7) | (22.0) |
| DSS + IIa (10 mg/kg) | 10 | 4.60 +/− 0.40** | 3.70 +/− 0.62* | 1.70 +/− 0.37 | 11.25 +/− 1.42** |
| Inhibition (%) | | (33.0) | (41.7) | (46.9) | (37.4) |
| DSS + IIa (30 mg/kg) | 9 | 4.70 +/− 0.36** | 3.94 +/− 0.62 | 1.67 +/− 0.44 | 11.70 +/− 1.32* |
| Inhibition (%) | | (31.6) | (38.0) | (47.8) | (34.9) |
| DSS+ Ia- (50 mg/kg) | 10 | 4.80 +/− 0.38** | 4.25 +/− 0.72 | 1.98 +/− 0.45 | 12.33 +/− 1.40* |
| Inhibition (%) | | (30.1) | (33.1) | (38.1) | (31.4) |

The data were represented as mean +/− SE.
n = 9-10.
*p < 0.05,
**p < 0.01 compared with 3% DSS + vehicle, Dunnett test.

### REFERENCES

1. Peet D J, Janowski B A, Mangelsdorf D J. The LXRs: a new class of oxysterol receptors. Curr Opin Genet Dev 1998; 8(5):571-5.
2. Apfel R, Benbrook D, Lernhardt E, Ortiz M A, Salbert G, Pfahl M. A novel orphan receptor specific for a subset of thyroid hormone-responsive elements and its interaction with the retinoidlthyroid hormone receptor subfamily. Mol Cell Biol 1994; 14(10):7025-35.
3. Teboul M, Enmark E, Li Q, Wikstrom A C, Pelto-Huikico M, Gustafsson J A. OR-1, a member of the nuclear receptor superfamily that interacts with the 9-cis-retinoic acid receptor. Proc Natl Acad Sci USA 1995; 92(6):2096-100.
4. Song C, Kokontis J M, Hiipakka R A, Liao S. Ubiquitous receptor: a receptor that modulates gene activation by retinoic acid and thyroid hormone receptors. Proc Natl Acad Sci USA 1994; 91(23):10809-13.
5. Willy P J, Umesono K, Ong E S, Evans R M, Heyman R A, Mangelsdorf D J. LXR, a nuclear receptor that defines a distinct retinoid response pathway. Genes Dev 1995; 9(9): 1033-45.
6. Peet D J, Turley S D, Ma W, Janowski B A, Lobaccaro J M, Hammer R E, et al. Cholesterol and bile acid metabolism are impaired in mice lacking the nuclear oxysterol receptor LXR alpha. Cell 1998; 93(5):693-704.
7. Luo Y, Tall A R. Sterol upregulation of human CETP expression in vitro and in transgenic mice by an LXR element. J Clin Invest 2000; 105(4):513-20. 22
8. Repa J J, Liang G, Ou J, Bashmakov Y, Lobaccaro J M, Shimomura I, et al. Regulation of mouse sterol regulatory element-binding protein-1c gene (SREBP-1c) by oxysterol receptors, alpha and LXRbeta. Genes Dev 2000; 14(22):2819-30.
9. Schultz J R, Tu H, Luk A, Repa J J, Medina J C, Li L, et al. Role of LXRs in control of lipogenesis. Genes Dev 2000; 14(22):2831-8.
10. Laffitte B A, Repa J J, Joseph S B, Wilpitz D C, Kast H R, Mangelsdorf D J, et al. LXRs control lipid-inducible expression of the apolipoprotein E gene in macrophages and adipocytes. Proc Natl Acad Sci USA 2001; 98(2):507-12.
11. Costet P, Luo Y, Wang N, Tall A R. Sterol-dependent transactivation of the ABC1 promoter by the liver X receptor/retinoid X receptor. J Biol Chem 2000; 275(36):28240-5.
12. Repa J J, Turley S D, Lobaccaro J A, Medina J, Li L, Lustig K, et al. Regulation of absorption and ABC1-mediated efflux of cholesterol by RXR heterodimers. Science 2000; 289(5484): 1524-9.
13. Venkateswaran A, Repa J J, Lobaccaro J M, Bronson A, Mangelsdorf D J, Edwards P A. Human white/murine ABC8 mRNA levels are highly induced in lipid-loaded macrophages. A transcriptional role for specific oxysterols. J Biol Chem 2000; 275(19):14700-7.
14 Venkateswaran A, Laffitte B A, Joseph S B, Mak P A, Wilpitz D C, Edwards P A, et al. Control of cellular cholesterol efflux by the nuclear oxysterol receptor LXR alpha. Proc Natl Acad Sci USA 2000; 97(22): 12097-102.
15. Schwartz K, Lawn R M, Wade D P. ABC1 gene expression and ApoA-1-mediated cholesterol efflux are regulated by LXR. Biochem Biophys Res Commun 2000; 274(3):794-802.
16. Repa J J, Mangelsdrof D J 2000 The role of orphan nuclear receptors in the regulation of cholesterol homeostasis. Annu Rev Cell Dev Biol 16:459-481.
   The above description fully discloses how to make and use the present invention. However, this invention is not limited to the particular embodiments described hereinabove, but includes all modification thereof within the scope of the appended claims and their equivalents. Those skilled in the art will recognize through routine experimentation that various changes and modifications can be made without departing from the scope of this invention. The various references to journals, patents and other patent applications that are cited herein are incorporated by reference herein as though fully set forth.

1. A method of treating or preventing IBD in a mammal; comprising, administering a therapeutically effective amount of LXR agonist, or a pharmaceutically acceptable salt, solvate, or physiologically functional derivative thereof.

2. The method of claim 1 in which IBD is selected from the group consisting of Crohn's disease, ulcerative colitis, and inflammatory colitis caused by bacteria, ischemia, radiation, drugs or chemical substances.

3. The method according to claim 1, wherein the LXR agonist is a compound of formula (II): 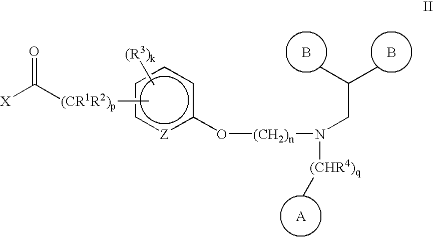
wherein:
X is OH or NH₂;
p is 0-6;
each R¹ and R² are the same or different and are each independently selected from the group consisting of H, C₁₋₈alkyl, C₁₋₈alkoxy and C₁₋₈thioalkyl;
Z is CH or N;
when Z is CH, k is 0-4;
when Z is N, k is 0-3;
each R³ is the same or different and is independently selected from the group consisting of
halo, —OH, C₁₋₈alkyl, C₂₋₈alkenyl, C₁₋₈alkoxy, C₂₋₈alkenyloxy, —S(O)ₐR⁶, —NR⁷R⁸, —COR⁶, COOR⁶, R¹⁰COOR⁶, OR¹⁰COOR⁶, CONR⁷R⁸, —OC(O)R⁹, —R¹⁰NR⁷R⁸, —OR¹⁰NR⁷R⁸, 5-6 membered heterocycle, nitro, and cyano;
a is 0, 1 or 2;
R⁶ is selected from the group consisting of H, C₁₋₈alkyl, C₁₋₈alkoxy and C₂₋₈alkenyl;
each R⁷ and R⁸ are the same or different and are each independently selected from the group consisting of H, C₁₋₈alkyl, C₂₋₈alkenyl, C₃₋₈alkynyl;
R⁹ is selected from the group consisting of H, C₁₋₈alkyl and —NR⁷R⁸;
R¹⁰ is C₁₋₈alkyl;
n is 2-8;
q is 0 or 1;
R⁴ is selected from the group consisting of H, C₁₋₈alkyl, C₁₋₈alkenyl, and alkenyloxy;
Ring A is selected from the group consisting of C₃₋₈cycloalkyl, aryl, 4-8 membered heterocycle, and 5-6 membered heteroaryl;
each ring B is the same or different and is independently selected from the group consisting of C₃₋₈cycloalkyl and aryl.

4. The method according to claim 3, in which the LXR agonist is the compound of formula (IIa)

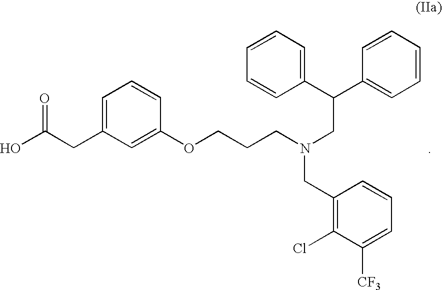

5. The method according to claim 1, wherein the LXR agonist is a compound of compounds of formula (I): 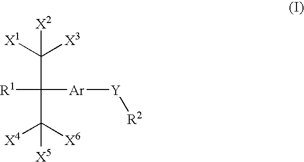
wherein:
Ar represents an aryl group; R¹ is —OH, —O—(C₁-C₇)alkyl, —OC(O)—(C₁-C₇)alkyl, —O—(C₁-C₇)heteroalkyl, —OC(O)—(C₁-C₇)heteroalkyl, —CO₂H, —NH₂, —NH(C₁-C₇)alkyl, —N((C₁-C₇)alkyl)₂ or —NH—S(O)₂—(C₁-C₅)alkyl;
R² is (C₁-C₇)alkyl, (C₁-C₇)heteroalkyl, aryl and aryl(C₁-C₇)alkyl;
X¹, X², X³, X⁴, X⁵ and X⁶ are each independently H, (C₁-C₅)alkyl, (C₁-C₅)hetroalkyl, F or Cl, with the proviso that no more than three of X¹ through X⁶ are H, (C₁-C₅)alkyl or (C₁-C₅)heteroalkyl; and
Y is —N(R¹²)S(O)ₘ—, —N(R¹²)S(O)ₘN(R¹³)—, —N(R¹²)C(O)—, —N(R¹²)C(O)N(R¹³)—, —N(R¹²)C(S)— or —N(R¹²)C(O)O—, wherein R12 and R13 are each independently hydrogen, (C₁-C₇)aryl, (C₁-C₇)heteroalkyl, aryl and aryl(C₁-C₇)alkyl, and optionally when Y is —N(R¹²)S(O)ₘ— or —N(R¹²)S(O)ₘN(R¹³)—, R¹² forms a five, six or seven-membered ring fused to Ar or to R² through covalent attachment to Ar or R², respectively. In the above Y groups, the subscript m is an integer of from 1 to 2.

6. The method according to claim 5, in which the LXR agonist is the compound of formula Ia [chemical structure]


SEQUENCE LISTING (4 sequences)

SEQ ID NO: 1 - DNA, 1344 bp, human
atgtccttgtggctgggggcccctgtgcctgacattcctcctgactctgcggtggagctgtggaagccaggcgcacaggatgcaagcagccaggcccagggaggcagcagctgcatcctcagagaggaagccaggatgccccactctgctgggggtactgcaggggtggggctggaggctgcagagcccacagccctgctcaccagggcagagcccccttcagaacccacagagatccgtccacaaaagcggaaaaaggggccagcccccaaaatgctggggaacgagctatgcagcgtgtgtggggacaaggcctcgggcttccactacaatgttctgagctgcgagggctgcaagggattcttccgccgcagcgtcatcaagggagcgcactacatctgccacagtggcggccactgccccatggacacctacatgcgtcgcaagtgccaggagtgtcggcttcgcaaatgccgtcaggctggcatgcgggaggagtgtgtcctgtcagaagaacagatccgcctgaagaaactgaagcggcaagaggaggaacaggctcatgccacatccttgccccccaggcgttcctcacccccccaaatcctgccccagctcagcccggaacaactgggcatgatcgagaagctcgtcgctgcccagcaacagtgtaaccggcgctccttttctgaccggcttcgagtcacgccttggcccatggcaccagatccccatagccgggaggcccgtcagcagcgctttgcccacttcactgagctggccatcgtctctgtgcaggagatagttgactttgctaaacagctacccggcttcctgcagctcagccgggaggaccagattgccctgctgaagacctctgcgatcgaggtgatgcttctggagacatctcggaggtacaaccctgggagtgagagtatcaccttcctcaaggatttcagttataaccgggaagactttgccaaagcagggctgcaagtggaattcatcaaccccatcttcgagttctccagggccatgaatgagctgcaactcaatgatgccgagtttgccttgctcattgctatcagcatcttctctgcagaccggcccaacgtgcaggaccagctccaggtggagaggctgcagcacacatatgtggaagccctgcatgcctacgtctccatccaccatccccatgaccgactgatgttcccacggatgctaatgaaactggtgagcctccggaccctgagcagcgtccactcagagcaagtgtttgcactgcgtctgcaggacaaaaagctcccaccgctgctctctgagatctgggatgtgcacgaatga

SEQ ID NO: 2 - protein, 447 aa, human
MSLWLGAPVPDIPPDSAVELWKPGAQDASSQAQGGSSCILREEARMPHSAGGTAGVGLEAAEPTALLTRAEPPSEPTEIRPQKRKKGPAPKMLGNELCSVCGDKASGFHYNVLSCEGCKGFFRRSVIKGAHYICHSGGHCPMDTYMRRKCQECRLRKCRQAGMREECVLSEEQIRLKKLKRQEEEQAHATSLPPRRSSPPQILPQLSPEQLGMIEKLVAAQQQCNRRSFSDRLRVTPWPMAPDPHSREARQQRFAHFTELAIVSVQEIVDFAKQLPGFLQLSREDQIALLKTSAIEVMLLETSRRYNPGSESITFLKDFSYNREDFAKAGLQVEFINPIFEFSRAMNELQLNDAEFALLIAISIFSADRPNVQDQLQVERLQHTYVEALHAYVSIHHPHDRLMFPRMLMKLVSLRTLSSVHSEQVFALRLQDKKLPPLLSEIWDVHE

SEQ ID NO: 3 - DNA, 1383 bp, human
atgtcctctcctaccacgagttccctggatacccccctgcctggaaatggcccccctcagcctggcgccccttcttcttcacccactgtaaaggaggagggtccggagccgtggcccgggggtccggaccctgatgtcccaggcactgatgaggccagctcagcctgcagcacagactgggtcatcccagatcccgaagaggaaccagagcgcaagcgaaagaagggcccagccccgaagatgctgggccacgagctttgccgtgtctgtggggacaaggcctccggcttccactacaacgtgctcagctgcgaaggctgcaagggcttcttccggcgcagtgtggtccgtggtggggccaggcgctatgcctgccggggtggcggaacctgccagatggacgctttcatgcggcgcaagtgccagcagtgccggctgcgcaagtgcaaggaggcagggatgagggagcagtgcgtcctttctgaagaacagatccggaagaagaagattcggaaacagcagcaggagtcacagtcacagtcgcagtcacctgtggggccgcagggcagcagcagctcagcctctgggcctggggcttcccctggtggatctgaggcaggcagccagggctccggggaaggcgagggtgtccagctaacagcggctcaagaactaatgatccagcagttggtggcggcccaactgcagtgcaacaaacgctccttctccgaccagcccaaagtcacgccctggcccctgggcgcagacccccagtcccgagatgcccgccagcaacgctttgcccacttcacggagctggccatcatctcagtccaggagatcgtggacttcgctaagcaagtgcctggtttcctgcagctgggccgggaggaccagatcgccctcctgaaggcatccactatcgagatcatgctgctagagacagccaggcgctacaaccacgagacagagtgtatcaccttcttgaaggacttcacctacagcaaggacgacttccaccgtgcaggcctgcaggtggagttcatcaaccccatcttcgagttctcgcgggccatgcggcggctgggcctggacgacgctgagtacgccctgctcatcgccatcaacatcttctcggccgaccggcccaacgtgcaggagccgggccgcgtggaggcgttgcagcagccctacgtggaggcgctgctgtcctacacgcgcatcaagaggccgcaggaccagctgcgcttcccgcgcatgctcatgaagctggtgagcctgcgcacgctgagctctgtgcactcggagcaggtcttcgccttgcggctccaggacaagaagctgccgcctctgctgtcggagatctgggacgtccacgagtga

SEQ ID NO: 4 - protein, 460 aa, human
MSSPTTSSLDTPLPGNGPPQPGAPSSSPTVKEEGPEPWPGGPDPDVPGTDEASSACSTDWVIPDPEEEPERKRKKGPAPKMLGHELCRVCGDKASGFHYNVLSCEGCKGFFRRSVVRGGARRYACRGGGTCQMDAFMRRKCQQCRLRKCKEAGMREQCVLSEEQIRKKKIRKQQQESQSQSQSPVGPQGSSSSASGPGASPGGSEAGSQGSGEGEGVQLTAAQELMIQQLVAAQLQCNKRSFSDQPKVTPWPLGADPQSRDARQQRFAHFTELAIISVQEIVDFAKQVPGFLQLGREDQIALLKASTIEIMLLETARRYNHETECITFLKDFTYSKDDFHRAGLQVEFINPIFEFSRAMRRLGLDDAEYALLIAINIFSADRPNVQEPGRVEALQQPYVEALLSYTRIKRPQDQLRFPRMLMKLVSLRTLSSVHSEQVFALRLQDKKLPPLLSEIWDVHE